(19) United States
(12) Patent Application Publication
(10) Pub. No.: US 2009/0081173 A1
(43) Pub. Date: Mar. 26, 2009

(54) METHODS AND COMPOSITIONS INVOLVING BACTERIOPHAGE ISOLATES

(75) Inventor: Philip Serwer, San Antonio, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 12/188,941

(22) Filed: Aug. 8, 2008

Related U.S. Application Data
(60) Provisional application No. 60/955,277, filed on Aug. 10, 2007.

(51) Int. Cl. A61K 35/74; A61K 35/76; C12N 7/00; C12Q 1/70

(52) U.S. Cl. 424/93.42; 424/93.6; 424/93.4; 424/93.43; 424/93.44; 424/93.461; 435/235.1; 435/5; 435/239

(57) ABSTRACT

Disclosed are methods of increasing the virulence of a bacteriophage, comprising contacting a bacteriophage with a composition comprising a bacterium or bacterial extract; and a polymer. Also disclosed are methods of propagating and isolating therapeutic bacteriophages that involve use of dilute polymer compositions. Also disclosed are pharmaceutical compositions the bacteriophage for which virulence has been increased by the methods set forth herein or which have been isolated by the methods set forth herein.

This application claims the benefit of priority to U.S. Application Ser. No. 60/955,277, filed Aug. 10, 2007, which is hereby incorporated by reference in its entirety.

This invention was made with government support under grant RO1 GM24365-27 awarded by the National Institutes of Health. The government has certain rights in the invention.

### BACKGROUND OF THE INVENTION

1. Field of the Invention

The present invention relates generally to the fields of virology and treatment of infectious diseases. More particularly, it concerns methods of increasing the virulence of bacteriphages, and methods of isolating therapeutic bacteriphages. Therapeutic bacteriophages cure or ameliorate disease caused by infection with bacteria.

2. Description of Related Art

Bacteriophages are viruses that infect bacteria Some phages, called lytic, kill bacteria. This killing is done with specificity and is the basic phenomenon on which the concept of phage therapy is built. Therefore, phages that possess a lytic life cycle are the most suitable candidates for phage therapy.

Phage therapy was first proposed in the 1920's. After the introduction of antibiotics in the 1940s, in the US and Western Europe, little attention was paid to this field of therapeutics. A major limitation was narrow host range for any one bacteriophage. Bacteriophage mixtures, or “cocktails,” were typically used.

Subsequently, extensive use of antibiotics led to an increase in the number of bacterial strains resistant to most or all available antibiotics, causing increasingly serious problems and raising widespread fears of return to a pre-antibiotic era of untreatable infections and epidemics. Bacteriophage therapy is attracting renewed attention in the West as a potential weapon against drug-resistant microbes and hard-to-treat infection, especially infections aggravated by the formation of biofilms.

In order to develop effective means of treating infectious disease using bacteriophage therapy, it is important to identify strains of sufficient virulence to be of therapeutic benefit. Methods of isolation and characterization of bacteriophages for this purpose have not been adequate. To detect bacteriophages, light (fluorescence) microscopy has recently become a procedure for counting virus particles, primarily bacteriophages, in the environment (Weinbauer, 2004; Winter et al., 2004; Williamson et al., 2005; Filippini et al., 2006) and more recently has been shown to reveal whether or not a bacteriophage aggregates while propagating (Serwer et al., 2007). The correlation of virus aggregation with phage therapy potential is not known. Electron microscopy-based evidence for bacteriophage aggregation has been found, for example, in the case of both P1-like bacteriophages (Ackermann et al., 1994) and three different recently isolated soil-borne bacteriophages (Serwer and Wang, 2005; Serwer et al., 2007a,b).

Aggregation can make a phage invisible to many procedures of detection. For example, when electron microscopy is used to characterize viruses, the specimen usually, but not always (Ackermann and DuBow, 1987, Chapter 6), is previously subjected to clarification, i.e., removal of bacteria and other greater than about 1 μm-sized objects, via either centrifugation or filtering (see Carlson, 2005; Filippini et al., 2006). This clarification, however, also removes some virus aggregates. When used without clarification of samples, fluorescence microscopy-based procedures define virus particles to be resolution-limited spots of nucleic acid-specific staining material bound to a filter (Noble and Fuhrman, 1998; Filippini et al., 2006). Virus aggregates larger than the resolution limit would, in this case, be misidentified as non-viral (see, for example, FIG. 1 of Noble and Fuhrman, 1998). In addition, particles are tethered to a substrate, i.e., they are not free in solution for further analysis. Thus, aggregating viruses are apparently often overlooked. They potentially contribute to both microbial ecology and microbe-based disease causation to an extent that is not yet appreciated.

Given the probable loss of virus aggregates during most detection procedures that include fractionation, in situ microscopy is needed for detecting virus aggregates, including aggregates of virulent bacteriophages of potential use in phage therapy. Though approximations of in situ electron microscopy by negative staining have been developed (reviewed in Ackermann and DuBow, 1987), true in situ electron microscopy has not been feasible because (1) the specimen must be dried in the case of procedures other than cryo-electro microscopy, (2) cryo-electron microscopy requires thin specimens (less than about 1 μm) possibly incompatible with large aggregates (reviewed in Jiang and Ludtke, 2005; Grunewald and Cyrklaff, 2006) and (3) all forms of high resolution electron microscopy require immobilization of particles in the specimen. Cryo-electron microscopy has not been used in situ to characterize virus aggregation.

In situ fluorescence microscopy is an alternative that has potential for bypassing the following limitations of an electron microscopy: loss of aggregates during purification, drying of the specimen and immobilization of virus particles during specimen preparation. However, in practice, success with in situ fluorescence microscopy depends on the solving of several problems. These problems include (1) preventing the obscuring of virus particles by background fluorescence, given that specimens are impure, (2) preventing excess photo-bleaching, (3) determining whether large bright objects (i.e., potential aggregates) are comprised of particles smaller than the resolution of the microscope (sub-resolution particles) and doing all this while (4) avoiding the removal of both bacteria and those other large (>˜1 μm) objects that interfere with detection.

Thus, there is the need for more effective means of (1) first finding potentially therapeutic phages, (2) then assessing lytic character and therapeutic potential and (3) increasing therapeutic potential. Some of the more effective phages may aggregate in the wild and, therefore, may not be observable with the traditional procedures. In situ fluorescence microscopy is potentially the most direct and useful procedure for determining whether any phage aggregates in situ.

### SUMMARY OF THE INVENTION

The present invention provides for methods of (1) improving the detection of lytic phages, (2) methods of propagating and isolating therapeutic bacteriophage and (3) increasing phage lytic potential.

More specifically, in certain embodiments, the invention is directed to methods of increasing the lytic character of a bacteriophage that involve contacting a bacteriophage with a composition comprising a polymer, wherein the lytic character of the bacteriophage is increased. In particular embodiments, the composition undergoes a phase separation after the bacteriophage is contacted with the composition. This phase separation can be detected by any method known to those of ordinary skill in the art, such as by fluorescence microscopy.

In particular embodiments, the composition comprises a bacterial extract. In further embodiments, the composition comprises bacteria. In particular embodiments, the bacteria are not pathogenic. In further embodiments, the composition comprises bacteria and a bacterial extract. The bacteria/extracts may be of any species, more likely a non-pathogenic species or strain, such as a Staphylococcal species or Streptococcal species. Non-limiting examples of bacteria include Staphylococcus epidermidis, Helicobacter pylori, Streptococcus mutans, Streptococcus oralis, Streptococcus parasanguis, Bacillus thuringiensis and Propionibacterium acnes. The bacterial extract may be obtained from any bacterial species, such as any of the aforementioned bacteria.

The polymer may be any polymer found to have capacity for increasing lytic character of the phage. Functional polymers include dilute (0.07%-0.2%) agarose. The bacterial cell provides additional polymer, thought to be β-hydroxybutyrate in the case of Bacillus thuringiensis (Serwer et al., 2007c). In particular embodiments, the polymer is a carbohydrate. Non-limiting examples of polymers contemplated for inclusion in the methods set forth herein include a polymer derived from agar, a dextran, a cyclodextran, a copolymer of poly-N-isopropylacrylamide, a methylcellulose, a chitosan, a collagen, a tri-block copolymer of poly(ethylene glycol)-poly(lactic-co-glycolic acid)-poly(ethylene glycol), a tri-block copolymer of poly(propylene glycol)-poly(ethylene glycol)-poly (propylene glycol), poly(N-isopropyl acrylamide, hyaluronic acid, alginate, carboxymethylcellulose, polyvinyl pyrrolidone, polyvinyl alcohol, a polyethylene glycol, a water-soluble polyacylamide, a substituted polyacrylamide, a polydimethylacrylamide, a polyvinyl pyrrolidone, gelatin, polyvinyl alcohol, polylysine, carageenan, and an analog thereof. The composition may comprise a mixture of two or more polymers.

In particular embodiments, the polymer is derived from agar. In specific embodiments, the polymer derived from agar is agarose.

The concentration of polymer in the composition may be any concentration, so long as this polymer promotes clearing of bacteriophage cultures. In particular embodiments, the concentration of polymer in the composition is about 0.001% to about 0.1%. In more particular embodiments, the concentration of polymer in the composition is about 0.01% to about 0.05%. In even more particular embodiments, the concentration of polymer in the composition is about 0.075% to about 0.2%. In particular embodiments, the polymer is agarose, and the concentration of polymer in the composition is about 0.075% to about 0.2%.

In certain embodiments, the method further includes assessing virulence of the bacteriophage following contacting of the composition with the bacteriophage. In embodiments of the present invention, virulence is increased relative to virulence of the bacteriphage prior to said contacting. “Virulence” of the bacteriophage is operationally defined as capacity to clear a culture of bacteria. Virulence of the bacteriophage can be assessed using any method known to those of ordinary skill in the art. For example, virulence may be assessed by visually detecting light scattering decrease following contacting of the bacteriophage with a bacterial cells. In some embodiments, the extent of lysis may be compared to bacterial cultures that have not been contacted with the composition. In some embodiments, virulence is measured by assessing response of a subject with an infection to treatment following administration of the bacteriophage to the subject. The subject may be a mammal. Non-limiting examples include a cow, a horse, a pig, a goat, or a human. For example, the subject may be a cow with an infection that is further defined as bovine mastitis. For example, the bovine mastitis may be caused by group B streptococci, Pseudomonas sp., or staphylococcal sp.

The present invention also generally pertains to methods of isolating a therapeutic bacteriophage from a bacteriophage-containing sample that include the steps of (a) placing a bacteriophage-containing sample (soil, for example) on a surface (such as a nutrient-containing surface) to form a lower layer; (b) contacting the bacteriophage-containing sample with a composition that includes a polymer, bacterial cells and nutrients to form an upper layer; and (c) removing a phage from a plaque that forms in the upper layer, wherein the plaque contains bacteriphages that caused formation of the plaque. Other aspects concern a method of isolating a therapeutic bacteriophage from a bacteriphage-containing sample, comprising the steps of: (a) placing a bacteriophage-containing sample on a surface; (b) contacting the bacteriophage-containing sample with a first composition comprising polymer to form a lower layer; (c) contacting the lower layer with a second composition comprising a polymer and a host bacteria to form an upper layer on top of the lower layer, wherein the upper layer forms a gel following contact; (d) removing a phage from a plaque that forms in either layer, wherein the plaque contains bacteriophages that caused formation of the plaque. In certain embodiments the method further includes inoculating the host bacteria with bacteriophage by creating a passageway between the lower layer and the upper layer following step (c).

The first composition may include any of the aforementioned polymers or a mixture of any of the aforementioned polymers. In some embodiments, the concentration of polymer in the composition is about 0.001%, 0.002%, 0.003%, 0.004%, 0.005%, 0.006%, 0.007%, 0.008%, 0.009%, 0.010%, 0.011%, 0.012%, 0.013%, 0.014%, 0.015%, 0.016%, 0.017%, 0.018%, 0.019%, 0.020%, 0.025%, 0.030%, 0.035%, 0.040%, 0.045%, 0.050%, 0.055%, 0.060%, 0.065%, 0.070%, 0.075%, 0.080%, 0.085%, 0.09%, 0.095%, 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 1.5%, 2.0%, 2.5%, 3.0%, 3.5%, 4.0%, 4.5%, or 5.0% or greater, or within any range of polymer concentrations derivable therein. In particular embodiments, the concentration of polymer is about 0.001% to about 0.10%. In further embodiments, the concentration of polymer in the first composition is about 0.01% to about 0.05%. In more particular embodiments, the concentration of polymer is 0.075% to about 0.2%.

In some embodiments, the upper and/or lower layer is further defined as a gel. The nutrient-containing surface may be comprised of a composition that includes one or more polymers. The concentration of polymer may be about 0.10%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0% or greater or any range derivable therein. In particular embodiments, the concentration of polymer in the lower layer is about 0.1% to about 1.0%. In more particular embodiments, the concentration of polymer in the lower layer is about 0.1% to about 0.5%. In still further embodiments, the concentration of polymer in the lower layer is 0.5% to about 2.0%.

The types of polymers in the upper and lower layer may be identical or different. In particular embodiments, at least one polymer derived from agar.

In particular embodiments, the upper and/or lower layer includes water (aqueous composition). The first and second compositions may include any of a number of additional components, such as tryptone, yeast extract, sodium chloride, casamino acids, glucose lactose, and other nutrients.

The upper layer may cover some or all or the lower layer. Thus, in some embodiments the upper layer may be discontinuous. The temperature of gelation is significant in that raising the temperature of gelation of agarose causes the gel pore sizes to get larger and, therefore, to accommodate larger viruses; the larger bacteriophages are currently very under-sampled and the use of these large pore gels should help to isolate more of them. The larger viruses have more coding capacity and, therefore, have more potential for growing on a wide range of hosts, important for phage therapy, as mentioned above.

In some embodiments, bacteriophage is harvested from the upper layers following a period of incubation. The period of incubation can be for any duration, but in particular embodiments it is between 12 and 20 hours. In some embodiments, a plaque is formed on the first and/or second layer, wherein the plaque is comprised of bacteriophages. Some or all of the plaque may be removed by any method known to those of ordinary skill in the art. For example, plaque material may be removed by scooping with a sterile spatula. Plaque material may be stored, such as by freezing. For example, freezing may be for long periods. For short periods (1-7 days), samples can be stored in the refrigerator at 4° C.; for longer periods, samples can be frozen in the presence of 3-10% dextran. The bacteriophage that is harvested may be harvested and stored with any amount of the first and/or second composition. Thus, for example, in some embodiments, the bacteriophage is harvested in gel that includes one or more polymers.

In some embodiments, the method further comprises preparing a pharmaceutical composition that includes bacteriphages which have been harvested from the culture. A pharmaceutical composition can be prepared in accordance with any method known to those of ordinary skill in the art. Additional information is discussed elsewhere in this specification. In some embodiments, most (greater than 99.99%) of the bacterial cells are lysed by a combination of treatments including (a) digestion with lysozyme, (b) exposure to non-ionic detergent (e.g., Triton X-100) and (c) freeze-thawing (Serwer, 2007c). Removing the bacteria might be considered important, especially if they are pathogenic themselves.

The bacteriophages that have been harvested can be applied in a wide variety of diseases and conditions. For example, they can be applied in the treatment of infectious diseases of plants, animals, and humans. In particular embodiments the animal is a mammal. Non-limiting examples of mammals include mice, rats, horses, cows, goats, sheep, and primates. In particular embodiments, the mammal is a cow with bovine mastitis. Examples of other diseases contemplated for treatment include wounds and systemic infections. In some embodiments, the bacteriophage are applied topically to the wound or site of infection. In particular embodiments, the bacteriophage are applied to the site of infection in a dilute gel; one advantage of the dilute gel is increasing of the lytic character of the phage. In a further particular embodiment, the bacteriphages set forth herein can be applied in the treatment of diseases of trees and vines. For example, one such disease is Pierce's disease of fruit trees and grape vines. Other bacterial infections of plants contemplated for treatment with the bacteriophage set forth herein include infections due to Erwinia, Xanthomonas and Pseudomonas.

The present invention also generally pertains to a method of treating or preventing an infectious disease in a subject, comprising contacting the subject with a pharmaceutically effective amount of a composition comprising a therapeutic bacteriophage that has been isolated in accordance with the methods set forth above. The subject may be a mammal as discussed above. In particular embodiments, the subject is a cow with bovine mastitis. In further specific embodiments, the subject is a human with an infectious disease, such as a bacterial disease. The composition may further comprise a polymer and bacteria or bacterial extract, as set forth above. In specific embodiments, the bacteriophage is 0305φ8-36. The composition may optionally include one or more additional components, such as a carrier, a polymer, or a secondary form of therapy for an infectious disease.

The present application also generally pertains to a method of treating or preventing an infectious disease in a subject, comprising contacting the subject with a composition comprising: (a) a bacteriophage and (b) a polymer to increase the virulence of the bacteriophage. In further embodiments the composition includes bacteria or bacterial extract. The bacterial extract may be derived from any non-pathogenic bacteria, including any of those specific examples of bacteria as discussed above. Similarly, the polymer may be any polymer, such as any of those examples of polymers discussed above. In particular embodiments, the polymer is agarose. In some embodiments, the concentration of polymer in the composition is about 0.075%, 0.080%, 0.085%, 0.09%, 0.095%, 0.1%, 0.11%, 0.12%, 0.13%, 0.14%, 0.15%, 0.16%, 0.17%, 0.18%, 0.19%, or 0.2% agarose by weight, or any range of agarose concentrations derivable therein. The subject may be any subject as discussed above. In particular embodiments, the subject is a human with a bacterial infection. For example, the infection may be due to Staphylococcus aureus, Staphylococcus epidermidis, Helicobacter pylori, Streptococcus pneumoniae, Streptococcus mutans, Streptococcus oralis, Streptococcus parasanguis, Streptococcus pyogenes, Streptococcus viridans, Group A streptococcus, anaerobic streptococcus, Hemophilus influenzae, Shigella dysenteriae, Mycobacterium tuberculosis, Mycobacterium leprae, Mycobacterium asiaticum, Mycobacterium intracellulare, Mycoplasma pneumoniae, Mycoplasma hominis, Neisseria meningitidis, Neisseria gonorrhea, Klebsiella pneumoniae, Pseudomonas aeruginosa, Propionibacterium acnes, Treponema pallidum, Treponema pertanue, Treponema carateum, Escherichia coli, Salmonella typhimurium, Borrelia burgdorferi, Leptospirex hemoragia, or Citrobacter fruendii. In other specific embodiments, the subject is a cow with bovine mastitis.

Aspects of the present invention concern pharmaceutical compositions that include: (a) a bacteriophage; and (b) a polymer. The composition may further include bacteria or bacterial extract. The bacteria, bacterial extract, and polymer can be any of those previously set forth. In particular embodiments the bacterial strain is Staphylococcus, Streptococcus, or Bacillus thuringiensis. In further embodiments the composition includes a bacterial extract derived from a Staphylococcus, a Streptococcus, or Bacillus thuringiensis.

The present invention also concerns pharmaceutical compositions comprising any of the bacteriophage set forth herein for which virulence has been increased or which have been identified by any of the methods set forth herein and a pharmaceutically acceptable carrier. In particular embodiments, the composition is an aqueous composition. The composition may comprise any of a number of additional components. Non-limiting examples are set forth in the sections that follow, and include one or more additional therapies such as antibiotics, and polymers as set forth above. A specific embodiment concerns a pharmaceutical composition that includes bacteriophage 0305φ8-36 or a 0305φ8-36 mutant and a pharmaceutically acceptable carrier. The mutant might be selected for either low or no aggregation.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1A, B, C. Observation of two phases when purified bacteriophage 0305φ8-36 particles are mixed with host cells. Purified bacteriophage 0305φ8-36 particles (2.0 μl) were mixed with host cells (2.7 μl). The host cells were grown in an upper layer of 0.075% agarose gel for 10.5 hr. This mixture was then DAPI-stained and prepared for fluorescence microscopy by the procedure in Example 1. The microscope was focused at the surface of the microscope slide. (A) A field with both weakly fluorescent (upper right and lower left) and darker phase is shown for bacteriophage 0305φ8-36. (B) The field of (A) is shown after applying pressure to the cover glass. (C) The field of (A) and (B) is shown after releasing pressure. The interfaces of the two phases are indicated with dashed lines. The arrows indicate bacteria. The length of the bar is 10 μm. This two phase behavior is likely to be the source of the increased lytic character of the bacteriophage.

FIG. 2A, 2B, 2C, 2D. Larger 0305φ8-36 aggregate observed by in situ fluorescence microscopy. A 10.5 hr. bacteriophage 0305φ8-36 plaque was dissected (the entire plaque, except for the border, was clear) and then stained with DAPI both unassisted and assisted by artificial lysis of residual cells and digestion of cellular nucleic acids. The surface of the microscope slide was imaged. (A) A field with a single large, “tufted” aggregate in the unassisted specimen is shown. (B) The field of (A) is shown after applying pressure to the cover glass. (C) The field of (A) and (B) is shown after releasing the pressure on the cover glass. (D) Another large aggregate is shown, but from the assisted specimen. In (A)-(C), the numbered arrows indicate sub-aggregates of resolution-limited particles. The dashed line outlines a tuft of the weakly fluorescent phase. The length of the bar is 10 μm.

FIG. 3A, 3B. Macroscopic bacteriophage aggregates in plaques. A post gelation-generated plaque was formed in a 0.075% agarose gel in a Petri plate. Images of the Petri plate are shown at (A) 32 hr. and (B) 54 hr. Arrowheads in (A) indicate isolated turbid and opaque zones; arrowheads in (B) indicate late appearing, larger turbid zones. The arrow in (A) indicates a collection of turbid zones concentrated near the edge of the plaque. The Petri plate is 8.4 cm in diameter.

FIGS. 4A, 4B, 4C, and 4D. A multi-μm-sized aggregate and smaller aggregates from a turbid zone of a 54 hr. 0305φ8-36 plaque. DAPI-stained specimens were prepared from a dissected turbid zone of a 54 hr. 0305φ8-36 plaque. Comparatively small aggregates are shown drifting in bulk solution at (A) time=0 and (B) time=2.03 sec. A comparatively planar, non-tumbling aggregate is indicated with a bracket in (A) and (B); smaller, tumbling aggregates are indicated with numbered arrows. In relation to the field of FIG. 6A, the field of FIG. 6B is shifted +7 μm horizontally and −5 μm vertically. In addition, an apparent multi-em-sized aggregate was observed at the surface of the microscope slide. The aggregate was observed (C) before and (D) after application of pressure to the cover glass. The “X” in (C) and (D) marks the approximate center of counterclockwise rotation for the bright part of the aggregate at the lower right; the arrow indicates the relative translational movement of this part of the aggregate. The dashed lines encircle a finger of aggregated resolution-limited particles most of which are not visibly in contact, but move together as a solid mass. The length of the bar is 10 μm.

FIG. 5. In-plaque virulence controls of Bacillus thuringiensis bacteriophage 0305φ8-36.

FIG. 6. Unusual biology of 0304φ8-36.

FIG. 7. Aggregates as seeds: Bull's eye plaques.

FIG. 8. Bacteriophage 0305φ8-36.

### DESCRIPTION OF ILLUSTRATIVE EMBODIMENTS

There are several basic reasons why the concept of bacteriophage therapy for human or veterinary use has not been reduced successfully to practice: (i) In some cases, the efficacy of phage therapy was shown to be marginal or even negligible; (ii) unacceptable toxic side effects have been observed, mainly due to the use of bacteriophage compositions contaminated with bacterial debris typically containing toxins; (iii) more economical, broader spectrum alternatives, such as conventional chemical antibiotics, were developed; (iv) phage-resistant bacterial strains arose after introduction of the phage; and (vi) the body removed or inactivated the phage before it arrived at a target site. The intensive research in the field of molecular biology, which employs bacteria and phages as a research tool, has advanced practical knowledge on bacterial-phage interactions and revealed new techniques that may be utilized to overcome at least some of the above described difficulties in the development of phage therapy as reliable therapeutic tool.

The present invention in part provides for methods of enhancing the virulence of bacteriophage, and methods of isolating new and unusual lytic phages for application, such as for therapeutic anti-bacterial applications. For example, in one aspect the inventor has found that use of dilute (semi-solid) agarose to support plaques produces clear plaques of B. thuringiensis phage 0305φ8-36. Attempts to grow 0305φ8-36 in liquid culture of the same composition yields phage production. However, cell lysis is never sufficient to perceptively lower the turbidity of liquid cultures and liquid enrichment culture (Carlson, 2005) is completely ineffective in isolating this phage from the environment. Aggregation of 0305φ8-36 occurs when it is growing in dilute gels and, aggregation that blocks phage infection explains the absence of visible host cell lysis in liquid culture via the hypothesis that as 0305φ8-36 becomes more concentrated, it undergoes aggregation and the aggregation prevents 0305φ8-36 from infecting additional cells. Further, the invention also concerns the finding that polymers can increase the lytic character of bacteriophages, which increases detectability of the bacteriophage as well as efficacy in bacteriophage therapy. These findings in turn have led to the identification of improved methods of bacteriophage isolation.

### A. HOST BACTERIA AND BACTERIA FOR PREPARATION OF EXTRACTS

The present invention contemplates use of any host bacterium that has stable reproduction and does not produce a toxin that has deleterious effects on the organism to be helped by phage therapy. Determination and selection of such bacteria is performed using techniques known to those skilled in the art and includes testing for the absence of toxins particularly: alpha toxin, beta toxin, delta toxin, gamma toxin, enterotoxins A, B, C, and D, Toxic Shock Syndrome Toxin (TSST), exfoliatins A and B, leukocidin, fatty acid modifying enzyme, and hemolysins. Testing also includes tests for stability and reproducibility of lysates generated by the phage-induced lysis, and long-term sensitivity of the bacterial strains.

Non-limiting examples of host bacteria include, inter alia, staphylococci, hemophili, helicobacter, mycobacterium, streptococci, neisseria, klebsiella, enterobacter, proteus, bacteroides, pseudomonas, borrelia, citrobacter, escherichia, salmonella, propionibacterium, treponema, shigella, enterococci and leptospirex. Preferably the microorganism includes, inter alia, Staphylococcus aureus, Staphylococcus epidermidis, Helicobacter pylori, Streptococcus pneumoniae, Streptococcus mutans, Streptococcus oralis, Streptococcus parasanguis, Streptococcus pyogenes, Streptococcus viridans, Group A streptococcus and anaerobic streptococcus, Hemophilus influenzae, Shigella dysenteriae, Mycobacterium tuberculosis, Mycobacterium leprae, Mycobacterium asiaticum, Mycobacterium intracellulare, Mycoplasma pneumoniae, Mycoplasma hominis, Neisseria meningitidis, Neisseria gonorrhea, Klebsiella pneumoniae, Pseudomonas aeruginosa, Propionibacterium acnes, Treponema pallidum, Treponema pertanue, Treponema carateum, Escherichia coli, Salmonella typhimurium, Borrelia burgdorferi, Leptospirex, such as Leptospirex hemoragia, Citrobacter fruendii. More preferably, the microorganism is selected from any one of staphylococci, streptococci, citrobacter, escherichia and klebsiella, and most preferably, the microorganism is selected from Staphylococcus aureus, Streptococcus pyogenes, Klebsielia oxytoca, Escherichia coli and Citrobacter fruendii.

The present invention further contemplates use of any bacteriophage that stably reproduces in a selected bacterial strain. Determination and selection of such bacteriophages include testing for long-term stability during storage, as well as reproducibility, host range and completeness of lysis induction. The host strain is a strain that is sensitive to the lytic properties of the bacteriophage.

Various mechanisms can be used to select bacteriophage compositions that are lytic and therefore suitable for use in phage therapy. For example, bacteriophages may be extracted from the environment, such as from a soil sample. This can be effected using methods known in the art. (See, American Type Culture Collection Catalogue Of Bacteria And Bacteriophages, 18^{th} Ed., 1992).

The specific bacteria-bacteriophage combination can then be further selected according to the intended use. For example, if the desired use is to provide therapy for staphylococcal infections, one or more strains of staphylococcal or related bacteria are used as the bacterial host organisms. In this same example, one or more bacteriophages that are specific for staphylococcal bacteria, or are at least capable of having a productive infection in staphylococcal bacteria, are used to create the staphylococcal lysate. Alternatively, one bacterial strain may be grown to produce a bacterial culture or bacterial broth and then separate aliquots of the bacterial culture are each infected with a different bacteriophage to create individual lysates. These individual lysates may be used individually or combined to form compositions. Another option is to use different bacterial strains and then infect each strain with the same bacteriophage to yield lysate compositions that can be used individually or combined to form compositions. Still alternatively, different bacterial stains that are each infected with different bacteriophages and the resulting lysates are used individually or in combination to form compositions.

Selected bacterial strains are purified, and colonies of the bacteria are grown using conventional methods. When a bacterial organism is identified from any of the aforementioned sources, it is preferably grown in pure culture and frozen for storage at about −20 degrees C., −70 degrees C. or −80 degree C. These organisms can be frozen using conventional methods. One example of a method for freezing bacterial samples includes preparing overnight cultures of bacterial isolates, and then adding 10% dextran 10. The storage tubes as exemplified by microtubes then can be labeled and frozen

### B. METHODS OF ISOLATION AND PROPAGATION OF BACTERIOPHAGE

In certain aspects, the present invention pertains to methods of isolation and propagation of bacteriophage. Isolation and propagation can be by any method known to those of ordinary skill in the art.

Host bacteria can be grown in accordance with any method known to those of ordinary skill in the art. For example, in one such method bacteria may be grown in the following broth: 10 g Bacto tryptone, 5 g KCl in 1000 ml water (growth medium). Growth medium was used in the lower agar layer of all Petri plates, as discussed below. Growth medium, supplemented post-autoclaving with 0.002 M autoclaved CaCl₂, may also be used in gelled layers poured above the lower gelled layer. Bacteriophage suspension buffer may be used for both purification of bacteriophages and storage of purified bacteriophages: 0.01 M Tris-Cl, pH 7.4, 0.01 M MgCl₂, 3% polyethylene glycol, molecular weight=4,000. Polyethylene glycol may be added to the bacteriophage suspension buffer to stabilize bacteriophages (see Serwer et al., 1983). To inhibit bacterial growth in bacteriophage preparations during storage, 0.01 M sodium azide may be added to the bacteriophage suspension buffer.

In one embodiment, the bacteria may be isolated from the environment by streaking for colony formation at room temperature (22±3 C) on a layer of pre-gelled 1.0% agar in growth medium. Bacterial strains may be subjected to multiple (e.g., three) consecutive single colony isolation/propagations before storage. Liquid cultures may be aerated via diffusion. Storage of bacteria may be achieved by, for example, platinum needle transfer to a sterile solution of 10% dextran 10,000 in growth medium and then freezing at −70° C.

1. Isolation and Storage of Unpurified Bacteriophages

In some embodiments, the bacteriophage to extract and propagate is obtained from a solid environmental sample. The following is a non-limiting example of one method that can be applied in isolating and propagating bacteriophage.

To extract and propagate bacteriophages from a solid environmental sample (soil, for example), initially the environmental sample may be placed in a Petri plate on the surface of a 1% agar gel cast in growth medium. Next, an upper layer mixture may be poured around the sample. For example, the upper layer mixture may be molten agarose (4 ml, held at 50° C.) with overnight liquid culture of the host (˜0.1 ml, added from a dropper bottle). Most of the environmental sample may be usually submerged. This upper layer mixture may be made to form a gel by incubation at room temperature. The agarose for the upper layer gel may be any agarose or variant thereof known to those of ordinary skill in the art. For example, the agarose for the upper layer gel may be Seakem 0.075%-0.4%. The Petri plate may then be incubated for 16-20 hours at room temperature. The result is a “primary culture” that has a bacterial lawn that may sometimes have zones of clearing.

Transfer to a gelled lower agar layer (such as with a platinum needle) may be used to clone and propagate bacteriophages. To provide host cells for the second growth, an upper layer molten agarose-host mixture (e.g., 4 ml molten agarose +0.1 ml overnight host culture, at 50° C.) may be poured on the bacteriophage-inoculated lower layer agar gel. To clone, the agar may be inoculated in the center; the molten agarose-host mixture quickly swirled to non-uniformly spread bacteriophages. The upper layer may be made to form a gel by incubation at room temperature. Incubation may be continued at room temperature for 16-20 hours, for example, to produce a “secondary culture”. Usually, some regions of the secondary culture have confluent lysis of the bacterial lawn. However, other regions almost always have single plaques in a secondary culture, because the bacteriophages may be spread non-uniformly. This procedure of single-plaque propagation will be called needle cloning.

Needle cloning of a bacteriophage may be continued until the bacteriophage has experienced at least three consecutive single-plaque isolations. Then, a sample of a single plaque is stored by, for example, (1) platinum needle transfer to a sterile solution of 10% dextran 10,000 in growth medium, and (2) freezing at −70° C. To document the plaques observed, either a digital or a conventional photograph of a Petri plate may be obtained during epi-illumination with white light. Plates with dilute (0.075-0.2%) upper layer agarose gels should be handled gently and not inverted. Upper layer gels with agarose concentrations of, for example, 0.075-0.2% are generally solid enough to support plaques, but not solid enough to maintain integrity when a plate was inverted. In other embodiments, hydrocolloid is used, such as at a concentration of 0.075-0.2%.

2. Preparative Growth and Purification of Bacteriophage Particles

Preparative growth and purification of bacteriophage particles may be by any method known to those of ordinary skill in the art. For example, for preparative in-gel growth of bacteriophage, by the above procedure of needle cloning (0.15-0.2% upper layer agarose gel) may be used with inoculation uniformly distributed around the lower layer agar gel. After incubation of the Petri plate, confluent lysis may be observed for either all or most of the bacterial lawn.

After preparative in-gel growth of a bacteriophage, the following procedure may be used to purify the progeny bacteriophage particles: First, the upper layer agarose gel is removed from a Petri plate. Bacteriophage particles in the pieces of upper layer gel are then extracted with the broth used for the upper layer gel. Then, the extracted bacteriophage suspension is clarified by pelleting of both cellular debris and pieces of gel at 3,000 rpm for 10 min in a JA-25.5 rotor used in a Beckman Avanti J-25 centrifuge at 4° C. The pellet may be twice extracted and clarified; the supernatants pooled and then re-clarified (“plate stock”).

For further purification, bacteriophages may then be pelleted by centrifugation of the pooled supernatants at 21,000 rpm for 1.0 hr in a JA-25.5 rotor at 4° C. The pelleted bacteriophage particles are then resuspended in bacteriophage suspension buffer. Purification may be completed using a cesium chloride gradient technique.

3. Monitoring of Bacteriophage Growth in Liquid Culture

To monitor the time course of bacteriophage growth in liquid culture, diluted bacteriophage particles may be mixed with a bacterial culture in growth medium. The infected culture may be incubated at 22° C. Cultures may be aerated by diffusion into a shallow culture, or aerated by use of forced air (e.g., a bubbler).

To determine the PFU in a plaque, a plaque-supporting piece of gel may be removed from a Petri plate with the wide end of a sterile Pasteur pipette (approximate plaque volume=0.1 ml). The piece of gel may be placed in 1 ml bacteriophage suspension buffer with 0.01 M sodium azide. Bacteriophage particles may be eluted with shaking at 4° C. for 8 hours. The PFU/ml of eluted bacteriophages may optionally determined.

4. Electron Microscopy

After purification, bacteriophage particles may be dialyzed against bacteriophage suspension buffer. The bacteriophage particles may be negatively stained with 1% uranyl acetate and observed by electron microscopy (Serwer, 1976).

### C. POLYMERS

Certain embodiments of the present invention pertain to methods of enhancing the virulence of a bacteriophage that involve contacting the bacteriophage with a composition comprising one or more polymers.

Non-limiting examples of polymers contemplated by the present invention include agarose, dextrans, cyclodextrans, copolymers of poly-N-isopropylacrylamide, polysaccharides, methylcellulose, a chitosan and beta-glycerophosphate solution, collagen, tri-block copolymer of poly(ethylene glycol)-poly(lactic-co-glycolic acid)-poly(ethylene glycol), tri-block copolymer of poly(propylene glycol)-poly(ethylene glycol)-poly (propylene glycol), poly(N-isopropyl acrylamide, hyaluronic acid, derivatives of hyaluronic acid, alginate, derivatives of alginate, carboxymethylcellulose, polyvinyl pyrrolidone, polyvinyl alcohol, polyethylene glycols, water-soluble polyacylamides, substituted polyacrylamides, polydimethylacrylamide, polyvinyl pyrrolidone, gelatin, polyvinyl alcohol, polylysine. and mixtures thereof.

Information regarding the preparation of agarose and agarose solutions is found in U.S. Patent Application Pub. No. 20050192249 and 20050267296, each of which is herein specifically incorporated by reference. In particular embodiments, the molecular weight of the polymer is preferably between about 2,000 Da and about 10,000,000 Da. In more particular embodiments, the molecular weight of the polymer is between about 100,000 Da and about 7,000,000 Da. In even more particular embodiments, the molecular weight of the polymer is between about 100,000 and 1,000,000.

Other examples of polymers for therapeutic applications are set forth in U.S. Pat. No. 6,335,035, U.S. Pat. No. 6,335,035, U.S. Pat. No. 6,716,251 U.S., U.S. Pat. No. 6,586,493, Patent App. Pub. No. 20060280797, U.S. Patent App. Pub. No. 20060118418 each of which is herein specifically incorporated by reference in its entirety.

### D. PHARMACEUTICAL PREPARATIONS AND TREATMENT OF DISEASE

### 1. Definitions

“Treatment” and “treating” as used herein refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

### 2. Pharmaceutical Compositions

Pharmaceutical compositions according to the present invention can be prepared by admixing a quantity of a bacteriophage stock composition with a pharmaceutically acceptable carrier. For example, the compositions of the present invention are administered in the form of topical compositions, but may also be developed to be injectable. A typical composition for such purpose comprises a pharmaceutically acceptable carrier. For instance, the composition may contain about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, or 1000 micrograms of the bacteriophage stock composition per milliliter of solution, or any range of concentration derivable therein. In some embodiments, the solution is a phosphate buffer containing NaCl and divalent cation, for example, 0.01 M Magnesium chloride. In some embodiments, the concentration of the bacteriphage is 10⁹-10¹³ particles/ml.

Other pharmaceutically acceptable carriers include aqueous solutions, non-toxic excipients, including salts, preservatives, buffers and the like, as described in Remington's Pharmaceutical Sciences, 15^{th} Ed. (1975) and The National Formulary XIV., 14th Ed. (1975), the contents of which are hereby incorporated by reference. Examples of non-aqueous solvents include propylene glycol, polyethylene glycol, vegetable oil and injectable organic esters such as ethyloleate. Aqueous carriers can include water, alcoholic/aqueous solutions, saline solutions, parenteral vehicles such as sodium chloride, Ringer's dextrose, and the like. Intravenous vehicles include fluid and nutrient replenishers. Preservatives include antimicrobials, anti-oxidants, and inert gases. The pH and exact concentration of the various components of the bacteriophage pharmaceutical compositions of the invention can be adjusted according to routine known in the art. See Goodman And Gilman's The Pharmacological Basis For Therapeutics (7^{th} Ed.).

Alternatively, the bacteriophage pharmaceutical compositions of the present invention can be in the form of liposomes, lipophilic microcapsules, dendrimers or the like for oral administration to treat systemic infections. Those skilled in the art are capable of preparing the bacteriophage compositions of the present invention in the form of a lipophilic microcapsule, a dendrimer or a liposome using conventional techniques known in the art. The skilled artisan also is capable of providing a bacteriophage composition that can be administered intranasally, rectally, transdermally, topically, or other known routes of administration of medicaments. In particular embodiments, the bacteriophage composition is formulated for topical application to a subject.

As discussed above, the bacteriophage compositions may optionally include any polymer as set forth above.

The compositions of the present invention can be used to treat mammals having an infectious disease, such as a bacterial infection. For example, the mammal may be a horse, a cow, a pig, or a human. In particular embodiments, the mammal is a human with an infectious disease. The infectious disease may be any infectious disease amenable to treatment with a bacteriophage composition as set forth herein. For example, the disease may be bovine mastitis.

Suitable bacteriophage-containing compositions are anticipated that will be effective in killing, obliterating or reducing the quantity of any of the bacterial microorganisms using the guidelines presented above. In particular embodiments, the bacteriophage is 0305φ8-36.

The compositions of the present invention preferably are administered topically, intravenously, intranasally, orally, etc., in an amount and for a period of time effective to treat the bacterial infection. The expression “treating bacterial infections,” as it is used throughout this description, denotes either (i) killing or obliterating sufficient bacterial microorganisms to render the microorganisms ineffective in infecting the host, or (ii) reducing a sufficient quantity of bacterial microorganisms so as the render the microorganisms more susceptible to either the immune system or treatment using conventional antibiotics. Determining an effective amount of host-specific, non-toxic purified bacteriophage composition to be administered in accordance with the present invention entails standard evaluations. An assessment in this regard would generate data concerning bioavailability, absorption, metabolism, serum and tissue levels and excretion, as well as microorganism levels, markers, and cultures. The appropriate dosage and duration of treatment can be ascertained by those skilled in the art using known techniques.

According to one embodiment, bacteriophage compositions prepared according to the present invention can be used to reduce but not entirely obliterate the population of harmful microorganisms, thereby rendering the infectious focus more susceptible to chemotherapeutic antibiotics and thus reducing, in combination therapy duration, side effects, and risks of the latter. Thus, the bacteriophage pharmaceutical compositions of the present invention can be used in combination with known antibiotics such as aminoglycosides, cephalosporins, macrolides, erythromycin, monobactams, penicillins, quinolones, sulfonamides, tetracycline, and various other anti-infective agents. Those skilled in the art can refer to the Physician's Desk Reference, 50^{th} Ed. (1996), or similar reference manuals for a more complete listing of known antibiotics which could be used in combination with the bacteriophage compositions. For example, a bacteriophage composition effective against various strains of staphylococcus could be used in combination with a cephalosporin such as Keflex™ or Keftab™ (both from Cephalexin). Those skilled in the art, using the guidelines provided herein, are capable of designing an effective treatment regimen by either using the bacteriophage composition alone or using a bacteriophage composition in combination with antibiotics.

In some embodiments, the bacteriophage of the present invention is dried. The dried preparation can be applied or administered to an organism in a dried form, or it can be reformulated in a carrier prior to administration. Such dried preparations simplify storage and shipping and, in a preferred embodiment, the temperature would be ambient temperature. Bacteriophage 0305φ8-36 was isolated from a dry soil sample from a region of daytime soil temperatures as high as 140° F.

### 3. Secondary Therapy

Certain embodiments of the present invention provide for the administration or application of one or more secondary forms of therapies for the treatment or prevention of an infectious disease.

The secondary form of therapy may be administration of one or more secondary pharmacological agents that can be applied in the treatment or prevention of an infectious disease. If the secondary therapy is a pharmacological agent, it may be administered prior to, concurrently, or following administration of the bacteriophage-containing composition.

Examples of secondary therapy include antibiotics, such as penicillins (including aminopenicillins and/or penicillinas in conjunction with penicillinase inhibitor and anti-fugal agents), cephalosporins (and the closely related cephamycins and carbapenems), fluoroquinolones, tetracyclines, macrolides, aminoglycosides. Specific examples include, but are not limited to, erythromycin, bacitracin zinc, polymyxin, polymyxin B sulfates, neomycin, gentamycin, tobramycin, gramicidin, ciprofloxacin, trimethoprim, ofloxacin, levofloxacin, gatifloxacin, moxifloxacin, norfloxacin, sodium sulfacetamide, chloramphenicol, tetracycline, azithromycin, clarithyromycin, trimethoprim sulfate and bacitracin.

The interval between the bacteriophage therapy and the secondary therapy may be any interval as determined by those of ordinary skill in the art. For example, the interval may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. In some embodiments, the timing of administration of a secondary therapeutic agent is determined based on the response of the subject to the bacteriophage therapy.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### In Situ Fluorescence Microscopy of Bacteriophage Aggregates

### Materials and Methods

Media and Buffers

The medium used for growing both environmental bacteriophages and their hosts was the following: 10 g Bacto tryptone, 5 g KCl in 1000 ml water with 0.002 M CaCl2 added post-autoclaving (growth medium). For supporting bacteriophage plaques, the upper gel used in Petri plates was made of agarose (Seakem Gold; Cambrex Corp., Walkersville, Md.; concentration in the text) dissolved in growth medium. Beneath the upper gel, the lower gel was made of 1% Sigma agar in 10 g Bacto tryptone, 5 g NaCl in 1000 ml water.

Hosts and Bacteriophages

Bacteriophages 0305φ8-36 (host=Bacillus thuringiensis; Serwer et al., 2007a, b) and G (host=Bacillus megaterium; Fangman, 1978) and their hosts are the strains previously described. Bacteriophage 0905φ8-18 was isolated de novo via overlay of a soil sample with a 0.15% agarose gel that contained the host, B. thuringiensis. Bacteriophage 0905φ8-18 was then clonally propagated and purified three times by needle transfer at room temperature (25±3° C.), as previously described (Serwer et al., 2004). Newly isolated bacteriophages are named by their month/year of isolation, followed by φ, followed by the host strain number, followed by a dash and then the number of the bacteriophage strain.

Purified bacteriophage 0305φ8-36 particles are aggregated, based on electron microscopy (Serwer et al. 2007a), sedimentation velocity during analytical ultracentrifugation (Serwer et al., 2007b) and arrest at the origin during non-denaturing agarose gel electrophoresis (unpublished observation). Purified bacteriophage 0905φ8-18 particles are not aggregated by the criterion of non-denaturing agarose gel electrophoresis; the results with 0905φ8-18 are qualitatively similar to those previously obtained (Wang et al., 2004) with other non-aggregating bacteriophages (unpublished observation). By electron microscopy (Serwer et al., 2007a), bacteriophage 0905φ8-18 is a siphovirus (non-contractile tail) with a polyhedral DNA-containing outer shell 27±2 nm in radius and a tail 162±12 nm long (not shown). Purified bacteriophage G also does not aggregate by the criterion of rate zonal centrifugation (Fangman, 1978).

Formation and Dissection of Bacteriophage Plaques for In Situ Fluorescence Microscopy

For in situ fluorescence microscopy of their contents, large (1-2 cm) nonoverlapping plaques were formed by use of the following two-step procedure: (1) An upper layer mixture of dilute (0.075%-0.1%) molten agarose and host cells was gelled at room temperature (25±3° C.) above a lower layer of 1.0% agar in a Petri plate. (2) Plaques were initiated by post-gelation needle inoculation at four symmetrically located places. Bacteriophages had previously been transferred to the needle by touching the clear region of a plaque. (3) The Petri plate was incubated at room temperature (post gelation-generated plaque).

A post gelation-generated plaque was dissected for fluorescence microscopy by pipeting of the dilute upper layer agarose gel. Care was taken to avoid removing the lower layer agar gel, because the lower layer agar gel is more concentrated and has a pore size small enough to perceptibly reduce the thermal motion of monomeric bacteriophage particles; the dilute upper layer agarose gel has an effective pore size too large to visibly alter the thermal motion of monomeric bacteriophage particles (Griess et al., 1989).

During dissection of a plaque, the upper layer agarose gel was liquid enough to be pipeted, but solid enough so that neighboring regions of the gel were not rearranged during pipeting. Comparatively large plaque size in 0.075-0.10% agarose gels assisted dissection. To observe plaque-associated bacteriophage particles with minimal presence of both residual host cells and host cell-associated nucleic acids, (1) residual host cells were burst by lysozyme/nonionic detergent/freeze-thaw and then (2) host cellular RNA and DNA were removed by DNase I and RNase A digestion, as previously described (50 μg/ml DNase I; Serwer et al., 2007c). When steps 1 and 2 followed plaque dissection before fluorescence microscopy, the in situ procedure is called “assisted”. The removal of host cells is beneficial for therapeutic applications, but is not required.

Staining and Fluorescence Microscopy

To DAPI-stain a specimen, the specimen (2.0 μl) was mixed with (1) β-mercaptoethanol (0.1 μl), (2) 15 μg/ml DAPI (4′, 6-diamidino-2-phenylindole; 0.2 μl) and (3) 0.2 M NaCl, 0.01 M Tris-Cl, pH 7.4, 0.001 M MgCl2 (2.7 μl). This mixture was incubated at room temperature for at least 1 min. before preparation for fluorescence microscopy. To ethidium-stain a specimen, two procedures were used: Procedure 1 was the staining procedure for DAPI with ethidium used instead of DAPI. Procedure 2 was Procedure 1 with 0.03 M sodium EDTA, pH 7.4 substituted for the magnesium containing buffer (third additive).

For all fluorescence microscopy, a ˜5.0 μl stained specimen was placed between a cover glass and glass microscope slide. Both cover glass and microscope slide had been cleaned by (1) soaking for 30 minutes in hydrogen peroxide (3% Topical Solution USP, Select Medical Products), (2) rinsing 5× with distilled water (3) soaking for 10 min. in acetone (greater than/equal to 99.5%, A.C.S. reagent, Sigma), (4) again rinsing 5× with distilled water and (5) hand drying with lens paper (VWR Scientific Products). The cleaned slides were stored covered at room temperature.

The specimen was visualized by use of an Olympus BX 60 fluorescence microscope (upright) with a short arc mercury vapor lamp and an oil immersion objective lens. A U-MNU filter set (Olympus; excitation filter wavelengths, 360-370 nm; longpass emission filter wavelengths, >420 nm; dichroic mirror, 400 nm) was used for DAPI-stained specimens; a U-MWIG filter set (Olympus; excitation filter wavelengths, 520-550 nm; long-pass emission filter wavelengths, >580 nm; dichroic mirror, 565 nm) was used for ethidium-stained specimens.

Real-time video of microscope images was recorded with a sensitive analog CCD camera (EXvision, SuperCircuits, Austin Tex.) connected to a VHS recorder (Panasonic AG-1980). Selected clips were converted to digital format using a Sony GV-300 mini DV recorder. The digital clips were imported to a Macintosh computer as QuickTime .mov files using iMovie software (Apple Corp., Cupertino, Calif.). All processing was done using the public domain software: ImageJ (Wayne Rasband, National Institutes of Health, USA). The .mov files were converted to 8-bit tif stacks by performing the operation: “Import/Using QuickTime”. Size was calibrated from a file of an image of a 600 line/mm grating captured with the same instrumentation. All operations, such as calibrating, cropping, and adjusting brightness/contrast, were applied to the entire stack. Selected frames from a stack were then saved and inserted into a PowerPoint (Microsoft Corp. Richmond Wash.) document for annotation of the final figures. Movie clips corresponding to some figures have been submitted in the on-line Supplementary Materials, as indicated in the text. These clips were created using the ImageJ operation “SaveAs/QuickTime”. The fields in the movies are basically the same as, but sometimes slightly larger than, the fields of the still images presented in the text.

To observe the changes induced by pressure-driven flow of a specimen, the cover glass was subjected to pressure near its center by pushing lightly with either the eraser end of a pencil or the objective lens of the microscope. To observe the effects of continuous, comparatively slow specimen flow, the specimen was observed in a region that had spontaneous, capillary-induced flow.

### Results

Solving the Problem of Background Fluorescence

Even when magnesium was chelated (Serwer et al., 2007c), background fluorescence was found to be a prohibitive problem when ethidium staining was used. This point is illustrated by the results obtained with an unassisted in situ dissection of a 0.1% agarose gel-supported plaque 54 hr. old (clear area of the plaque, as described below). When the microscope was focused on the surface of the cover glass, the result was observation of some immobile particles bound to the surface of the cover glass where the background fluorescence was comparatively low; no moving particles, either in or out of focus, were observed. The particles are likely to be either monomeric bacteriophage particles or small aggregates. No clear images of anything were obtained when the microscope was focused on any part of either the bulk liquid in the specimen or the microscope slide (not shown). This loss of definition was caused primarily by dramatic increase in background fluorescence that occurred as the microscope focused further into the specimen.

Resolution-limited, DAPI-stained particles were present in the hundreds/field when the microscope was focused at the surface of either the cover glass or the microscope slide. Some resolution-limited particles observed were undergoing thermal motion in bulk solution and, therefore, were usually not precisely in focus. A minority were immobile and bound to the cover glass. The resolution-limited particles were either monomeric bacteriophage particles or small bacteriophage particle aggregates, based on their absence when lawns of uninfected cells were examined.

The DAPI-stained specimen also had larger particles; some were bacteria with a sharp boundary. Others were bacteria with a boundary that was diffuse because the bacteria were out of focus, as found by bringing the particle into better focus. Other particles with a diffuse boundary were out of focus bacteriophage aggregates.

Fluorescence Microscopy of a Mixture of Bacteriophages and Host Cells

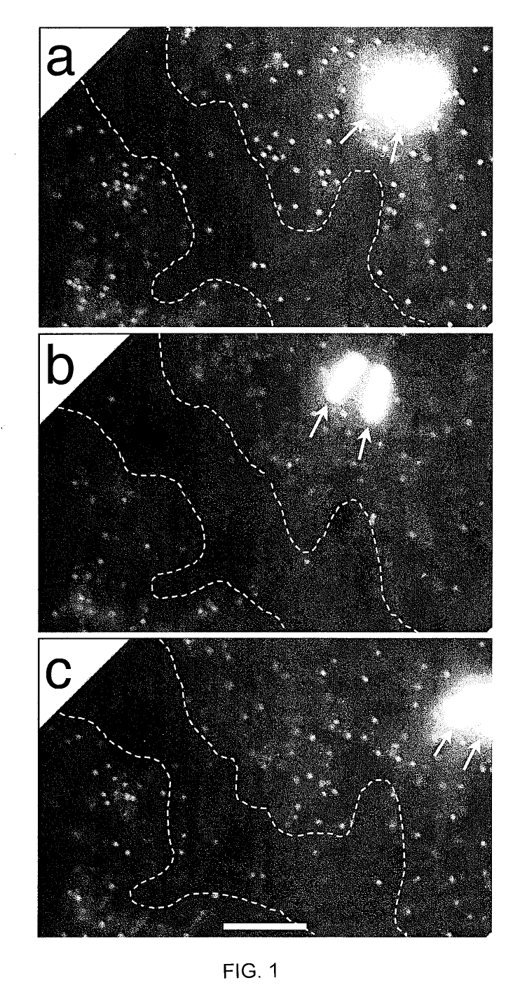

To assist interpretation of images obtained by in situ fluorescence microscopy, purified bacteriophage 0305φ8-36 particles were mixed with uninfected bacteria and, then, the mixture was immediately observed by fluorescence microscopy. The bacteria had been grown in the upper layer 0.075% agarose gel of a Petri plate that had not been bacteriophage inoculated. The result was that resolution-limited particles (i.e., either monomeric bacteriophage particles or small bacteriophage aggregates) undergoing thermal motion were observed in the 0305φ8-36/host mixture, but, surprisingly, appeared partitioned in two phases: (1) a weakly fluorescent phase in which resolution-limited particles selectively partitioned and (2) a darker phase. A representative field is in FIG. 1A; the microscope is focused on the microscope slide. Some motionless particles are bound to the microscope slide beneath both phases. This field has most of its weakly fluorescent phase at the top-right and some at the bottom-left. The darker phase is in between. Dashed lines indicate the two borders; these borders are not sharp. The weakly fluorescent phase has two overlapping bacteria in the top-right portion (arrows in FIG. 1A), as well as numerous thermally moving, resolution-limited particles in both the top-right and bottom-left portions.

The thermal motion of particles in the weakly fluorescent phase caused these particles to approach the inter-phase border, but thermal motion almost never carried the particles through the inter-phase border (<5% of border approaches). Over 2,000 approaches to the border from both sides were observed in drawing this conclusion. That is to say, the weakly fluorescent phase acted as a bacteriophage container. The darker phase was liquid (not air), based on (1) its low fluorescence in comparison the fluorescence of dried areas of the specimen, (2) its shape, including formation of narrow fingers such as the finger in FIG. 1A and (3) the presence in the darker phase of some thermally moving particles.

The thermal motion of the resolution-limited particles in the weakly fluorescent phase was visibly lower than the thermal motion in the darker phase. When the plane of focus was varied, the weakly fluorescent phase was sometimes found at the surface of the microscope slide while the apparently less dense darker phase floated above the weakly fluorescent phase.

To further test for the presence of two phases in the specimen of FIG. 1A, pressure was applied to cover glass. The pressure caused the weakly fluorescent phase to undergo deformation to a variable extent; the top-right portion deformed much more than the bottom left portion (FIG. 1B). The deformation included both change in the position of the borders (dashed lines in FIG. 1B) and separation of the superimposed bacteria (arrows in FIG. 1B). When the pressure was released, the weakly fluorescent phase underwent partial elastic return toward its original shape (FIG. 1C). Typically, the weakly fluorescent phase was irreversibly deformed and release of pressure did not completely return it to the original position and shape. However, resolution-limited particles did not cross the inter-phase border either during or after the deformation. Thus, the presence of two phases is confirmed. The chemistry of the two phases is not known beyond the presence of agarose and bacteriophage particles. Differences in deformability suggest nonuniformity of structure. The cause of the fluorescence of the weakly fluorescent phase is, at least in part, the presence of out of focus resolution-limited particles. Some of the fluorescence may also be from DNA expelled from either host cells or bacteriophage particles.

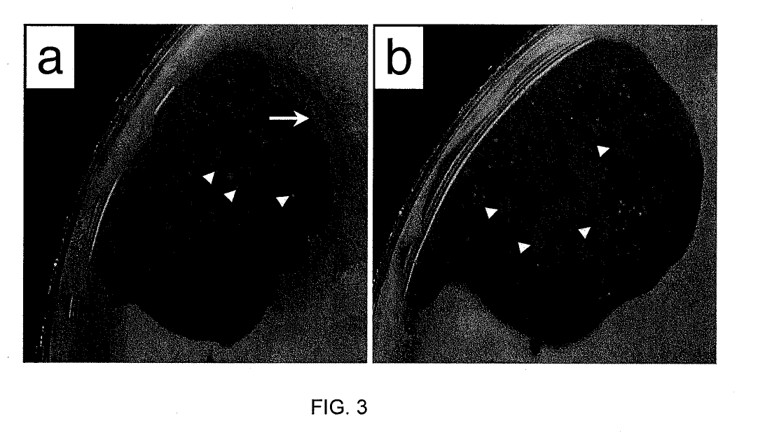

The phase separation of FIG. 3A occurred in less than 1 min. and was not observed when the following controls were performed: (1) The bacteria were observed without adding the bacteriophage particles. (2) The bacteriophage preparation was observed without adding the bacteria-containing upper layer agarose gel (not shown). The specimen preparation procedure for the controls was the same as that used for FIG. 3A. Thus, presence of two phases required both bacteriophage and bacteria. Presence of two phases also required agarose because the two phases were not observed when a concentrated liquid, agarose-free bacterial culture (˜109 bacteria/ml), instead of the upper layer bacteria-containing agarose gel, was mixed with bacteriophage particles. Two phases were not formed when either purified bacteriophage G or purified bacteriophage 0905φ8-18 was substituted for purified bacteriophage 0305φ8-36 (not shown).

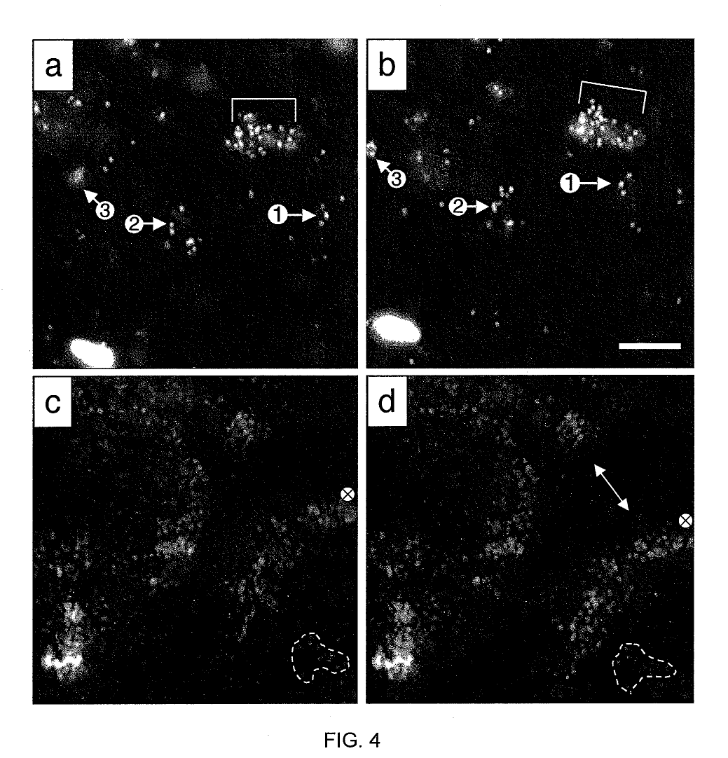

Most particles observed in FIG. 3 (A-C) were resolution-limited and, therefore, were either bacteriophage monomers or small aggregates. However, some resolution-limited particles in this specimen were aggregated with each other, as shown by tracking the particles through several frames. The typical number of resolution-limited particles per aggregate was 3-20. Most aggregates tumbled during tracking. Because of the difficulty in interpreting still images of a particle that is tumbling, images of the aggregates are not presented here; an example of aggregate tracking is presented below (see FIG. 4A,B) for a mobile aggregate that was, however, inhibited from tumbling. Finding of aggregates was expected for 0305φ8-36 because 0305φ8-36 is known to aggregate when purified.

In situ fluorescence microscopy of 0305φ8-36 Plaques

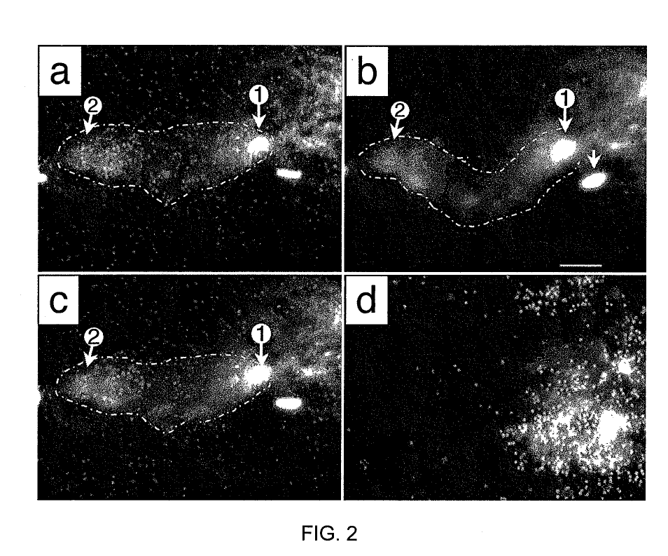

To observe the bacteriophage particles in a 0305φ8-36 plaque (in situ), initially the clear area of a 10.5 hr.-incubated 0305φ8-36 plaque was dissected and observed; the entire plaque was clear, except for the border. Like the bacteriophage/host cell mixtures in FIG. 1, an unassisted specimen had two-phase character. Again, aggregates of resolution-limited particles were observed in the weakly fluorescent phase (not shown). However, this specimen also had aggregates much larger and more complex than the aggregates in the specimen of FIG. 1. These latter aggregates were observed only at the surface of the microscope slide, not at the surface of the cover glass and not in bulk solution. An example is shown in FIG. 2A. This aggregate consists of (1) a many bacteriophage particle, bright sub-aggregate at the right (indicated with arrow 1), (2) a bridge formed by an apparent tuft of the weakly fluorescent phase in the region indicated by the dashed brackets and (3) an additional, less bright, many-particle sub-aggregate at the left (indicated by arrow 2). Some resolution-limited particles underwent limited, tethered thermal motion within aggregates of this type.

To test for the proposed tuft of the weakly fluorescent phase, pressure was applied to the cover glass. The pressure caused increase in motion of particles not attached to the aggregate (FIG. 2B) and also confirmed the presence of the weakly fluorescent tuft by making the weakly fluorescent zone bend downward while it remained attached to the aggregate (dashed lines in FIG. 2B). Most particles in the sub-aggregates marked 1 and 2 did not move during application of pressure. Release of the pressure caused the weakly fluorescent tuft to return to its original position (dashed lines in FIG. 2C). Thus, the aggregate of FIG. 2A-C consists of both (1) subaggregates of bacteriophage particles and (2) entangled, mobile material of the weakly fluorescent phase.

Aggregates as large as the aggregate in FIG. 2A-C were also seen in assisted specimens. Thus, formation of these aggregates did not require the presence of either host cells or nucleic acid. An example of such an aggregate from an assisted specimen of a 10.5 hr. plaque is in FIG. 2D.

Macroscopically Observed Effects of Plaque Maturation

When post gelation-generated 0305φ8-36 plaques were incubated for 16-32 hr., the aggregates observed in the clear, 10.5 hr. plaques became large enough to be visible by eye. Both turbid and opaque spots were seen (macroscopically) within a clear plaque at 32 hr. of incubation (FIG. 3A); arrowheads in FIG. 3A indicate some of these spots. In addition, these turbid and opaque spots sometimes were clustered, as in the zone indicated by an arrow in FIG. 3A. Fluorescence microscopy of an unassisted specimen of dissected turbid and opaque spots revealed the primary constituent to be bacteriophage aggregates, though a few bacteria were present. The concentrations of bacteria were below 10⁷ per ml, not concentrated enough to be the source of the light scattering observed visually.

In confirmation, some turbid and opaque spots dissipated when a plaque further matured to 54 hr. (FIG. 3B). This point is most dramatically illustrated in the region indicated by an arrow in FIG. 3A. New turbid and opaque spots formed in FIG. 3B; arrowheads indicate some of them. Colonies of bacteriophage-resistant bacteria do not auto-dissipate in the case of known bacteriophage plaques, to the authors' knowledge.

The following observations further confirm that the turbid and opaque zones are not bacterial colonies: (1) The macroscopic turbid and opaque zones had some macroscopic elasticity, perceived during pipeting of the material in these zones for specimen preparation. In contrast, both bacterial lawn and bacterial colonies were macroscopically viscous, not elastic. However, microscopically, bacteriophage aggregates from the turbid and opaque zones did have elastic character, as found in Serwer et al., 2007c); (2) In a 0.075% upper layer agarose gel on a Petri plate, the opaque and especially the turbid zones differed visually from bacterial colonies in that they were often flatter and were sometimes completely embedded in the agarose gel used to support a plaque.

Microscopically Observed Effects of Plaque Maturation

After a plaque matured to 32 hr., some aggregates observed by fluorescence microscopy continued to have the characteristics of the aggregates in FIG. 2, in specimens dissected from both the clear and opaque/turbid zones. However, a turbid zone of the 32 hr. plaque also had resolution-limited particles in aggregates that were not observed in the specimens of FIGS. 1 and 2. These aggregates were observed at the surface of the microscope slide, but not the surface of the cover glass. They contained both immobile resolution-limited particles (no thermal motion) and resolution-limited particles that (1) underwent detectable thermal motion that was, however, much lower than the thermal motion in either phase of bulk solution, but (2) usually were not in contact with each other. The lowered thermal motion of the latter particles indicates their trapping in a matrix that has viscosity above that of the bulk solution.

In support of matrix embedding, applying pressure to the cover glass caused almost all particles to move, but primarily in the plane of observation, as judged by the fact that the particles remained in focus. The motion was variable among the particles. In contrast, resolution-limited particles at the surface of the cover glass were immobile and remained immobile when pressure was applied; these particles are presumably glass-bound. Thus, motion in a plane implies an elastic matrix that held the particles in this plane.

To further test for elastic connection between the moving particles, the pressure was released and the response of the particles observed. The mobile particles responded by returning to a position close to the position before pressure was applied. This observation confirms that the particles were embedded in a matrix that had some elastic character. Close inspection reveals a gellike matrix in the background. An assisted specimen was used to (1) avoid effects caused by the presence of both bacteria and unpackaged nucleic acids and (2) reduce background when imaging near the surface of the microscope slide.

Effects of Further Plaque Maturation

Large aggregates resembling those in FIG. 2 were again observed when the surface of the microscope slide (but not the surface of the cover glass) was in focus for either an assisted or an unassisted specimen of dissected clear or dissected turbid zone from a plaque that had further matured to 54 hr. However, the glass-associated, dispersed, elastic aggregates were not found at 54 hr. In addition, focusing in bulk solution produced images that were richer in smaller aggregates than were comparable fields at earlier times. Some of these “solution” aggregates were comparatively large and planar. Being large and planar made the aggregates comparatively easy to perceive in a sequence of still images, because these large, planar aggregates did not tumble in solution. Also, visualization was assisted by the absence of a background from the dispersed, elastic aggregates. A drifting, largely planar “solution” aggregate of resolution-limited particles from a 54 hr. plaque (turbid region) is indicated with a bracket in FIG. 4A (time=0). The same aggregate is shown in FIG. 4B after drifting for 2.03 sec; the field was shifted for FIG. 4B (Legend to FIG. 4) to retain the particles present in FIG. 4A. Both tumbling and internal motion were small enough so that the basic structure of this aggregate is appreciated in just the two frames presented. Other, tumbling aggregates are indicated with numbered arrows in FIGS. 4A and 4B. Though comparatively low, the internal thermal motion was, however, not zero for the aggregate indicated by a bracket in FIGS. 4A and 4B.

As anticipated, multi-μm-sized aggregates were present at the surface of the microscope slide and only there, in specimens of a dissected 54 hr. plaque. These aggregates were even larger than the aggregates seen in 10 and 32 hr. specimens (FIG. 4C). The 54 hr. aggregates from assisted specimens were often over 10 μm across. Presence of these and other aggregates (above) only at the surface of the microscope slide is explained by the assumption that the aggregates had undergone 1-g sedimentation in the specimen. Essentially all resolution-limited particles (>99%) were immobile within the 54 hr. aggregates.

Characteristics of Large-Scale Aggregates

In the absence of moving particles in an apparent aggregate, binding of resolution-limited particles to sticky patches on the microscope slide is a possible, though unlikely, source of aggregation. To rigorously discriminate between classical aggregation and glass support binding of resolution-limited particles, again, pressure was applied to the cover glass. The result was that particles covering an entire multi-μm sized region moved with co-ordination as though linked to form a single, elastic object. This point is illustrated by the images both before (FIG. 4C) and after (FIG. 4D) applying pressure. The rightmost portion of the aggregate in FIG. 4C, 4D rotated as a rigid body around a point indicated by an X and this aggregate also moved translationally in the direction of the line in FIG. 4C, D. Release of pressure resulted in elastic return of the aggregate to its original state. Thus, at the pivot point, the aggregate had elastic character similar to that of the less dense aggregate.

In addition, a finger of the aggregate in FIGS. 4C, 4D (encircled by a dashed line) also rotated and recoiled, as a rigid body, in response to pressure. This finger has resolution-limited particles that are not in contact, including the lowest particle which is separated by more than 3 μm from the nearest resolution-limited particle in the rigid finger. As discussed below, aggregation-at-an-apparent-distance of this magnitude implies that some components of the aggregate are not associated with purified bacteriophage particles. The aggregate of FIGS. 4C, 4D appears to be a descendant of an aggregate like the less dense, elastic aggregate. The test of FIGS. 4C, 4D was performed over 100 times and all with the same basic result. All 0305φ8-36 apparent multi-μm-sized aggregates were classical aggregates.

Sometimes, an entire aggregate lifted away from the cover glass surface during application of pressure. The aggregate then fell at 1-g back to the surface (not shown). Thus, aggregates sometimes were not bound to the surface at even one point. In addition, this observation confirms that multi μm-sized aggregates, in general, had migrated to the microscope slide surface by gravitational sedimentation at 1-g.

Effects of the illumination of the microscope and dilution of the specimen The success of DAPI staining was initially counter-expectation because DAPI is known for comparatively high photobleaching when used to stain nucleic acids for fluorescence microscopy (Bustamante, 1991). During the present study, photobleaching was variable in specimens of all types. However, the stained, resolution-limited particles of unassisted in situ specimens consistently had less photobleaching than specimens of purified particles; sometimes photobleaching was undetectable for times over two minutes. Photobleaching was again variable, but typically higher for assisted specimens, as though the residual bacteria had reduced the photobleaching in the unassisted specimens. Half-lives for photobleaching of the immobile resolution-limited particles were often between 30 sec and 150 sec. in assisted specimens.

Unpurified 0305φ8-36 particles from preparative Petri plate lysates have a specific infectivity of >0.5 (Serwer et al., 2007a). This observation implies that aggregates have, at most, only a minor percentage of the total monomers after dilution for infectivity assay. To test for dilution-induced disaggregation of the undiluted aggregates observed by fluorescence microscopy, a dissection of a turbid zone of a 32 hr. plaque was diluted by a factor of 10 in growth medium and observed 30 min. later by fluorescence microscopy. From the start of observation, dissociation was observed via the absence of aggregates like those in FIG. 2 and FIGS. 4C, 4D. Resolution-limited particles were present at concentrations higher than what was expected for a 10× dilution. That is to say, 10× dilution dissociated the aggregates.

In addition, dilution increased the level of photo damage. The photo damage now was extensive enough so that it caused expulsion of DNA from the bacteriophage particles. Expulsion of DNA was initially observed via halos that surrounded the resolution-limited particles (not shown). Expulsion of DNA was dramatically confirmed by pressing on the cover glass and thereby stretching DNA molecules. The stretching was observed via the appearance of multi-micron-long fibers in both assisted and unassisted specimens (not shown). The “DNA-stretching” effect of pressing on the cover glass has previously been demonstrated; the longest molecules observed provide a minimal estimate of the length of genome (Serwer et al., 1995).

Dilution-induced dissociation can reveal the presence of aggregates when resolution-limited particles are so closely packed within aggregates that they are not separately resolved. Thus, dilution-induced dissociation is potentially a means for identifying viral aggregates in general, even when separate particles are not resolved within an aggregate.

Results with Non-Aggregating Bacteriophages. 0905φ8-18 and G

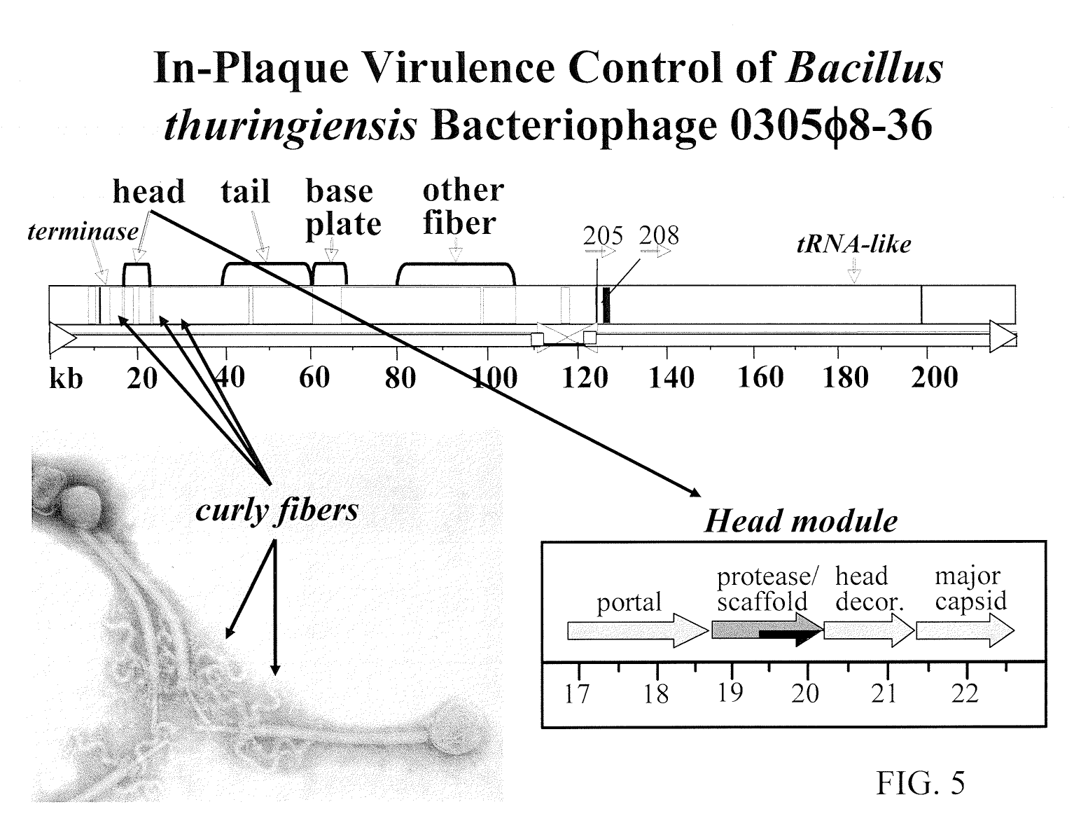

In the case of bacteriophage 0905φ8-18, a bacteriophage non-aggregating when purified (Materials and Methods Section), the massive aggregates of FIGS. 2 and 4C, 4D were completely absent from dissections of 1.5-2.5 cm, 20 hr. plaques when the dissections were observed by in situ fluorescence microscopy. However, the following were present: (1) the two phases illustrated in FIG. 1 for 0305φ8-36 (FIG. 5; arrowheads indicate immobile particles in the darker phase), even though two phases were not observed above when purified bacteriophage 0905φ8-18 particles were mixed with bacteria and (2) elastic, low density aggregates. The latter were, however, only occasionally present and were not as omnipresent as they were in the 32 hr. specimen of 0305φ8-36. The “in situ” concentration of 0905φ8-18 particles was higher than the “in situ” concentration of 0305φ8-36 particles.

In the case of bacteriophage G, a bacteriophage also non-aggregating when purified, the massive aggregates of FIG. 2 and FIGS. 4C, 4D were also completely absent from plaques. In addition, elastic, low-density aggregates were completely absent from plaques of bacteriophage G. Thus, the presence of the massive aggregates cleanly distinguishes 0305φ8-36 from both 0905φ8-18 and G and does so in agreement with the analysis of aggregation previously obtained for purified bacteriophage particles.

Further Observations on Discriminating Aggregating from Non-Aggregating Bacteriophages

To further understand the extent of bacteriophage aggregation, plaques were incubated for up to 96 hr. During this entire time, no separate plaque-associated turbid and opaque zones were formed by bacteriophage G, though the plaques did become turbid. However, macroscopic, turbid, gel-embedded, sometimes auto-dissipating zones did sometimes appear for 0905φ8-18 by 24 hr. Microscopy of the dissected, turbid 0905φ8-18 zones revealed the presence of bacteriophage 0905φ8-18 particles enmeshed in material like the material that formed the tuft of weakly fluorescent phase described above and illustrated in FIG. 2. The resolution-limited particles were not associated with each other either when enmeshed or when in solution (not shown). That is to say, 0905φ8-18 particles were resolution-limited in size. Thus, the constituents of the turbid 0905φ8-18 zones were cleanly distinguished by fluorescence microscopy from those of mature 0305φ8-36, although they were macroscopically more difficult to distinguish.

In summary, fluorescence microscopy was the key procedure for classifying bacteriophages by in situ aggregation state, though visual examination of plaques helps. Without the fluorescence microscopy, the turbid and opaque regions of both 0305φ8-36 25 and 0905φ8-18 were easily mistaken for bacterial colonies. If this had occurred, the entire in situ aggregation phenomenon would have been missed. Additional information regarding these findings can be found in Serwer et al., 2007d.

### Example 2

### In-Plaque Virulence Control of Bacillus Thuringiensis Bacteriphage 0305φ8-36

Bacillus thuringiensis bacteriophage 0305φ8-36 is lytic, based on both genomic sequence and formation of clear plaques in dilute (0.075-0.10%) agarose gels. However, 0305φ8-36 does not clear liquid cultures (Serwer, P. et al. Virol. J. 4, 21). In the present study, in situ fluorescence microscopy reveals that the dilute gel-supported 0305φ8-36 plaques have two phases. One is weakly fluorescent; the other is comparatively dark. Most bacteriophage particles are in the weakly fluorescent phase. Empirically, formation of two phases requires the presence of bacteriophage particles, bacteria and dilute agarose. The weakly fluorescent phase has both a mass density and a viscosity greater than those of the darker phase. Bacteriophage particles do not cross the phase boundary, even when the specimen is distorted by pressing on the cover glass.

The above data imply that both bacteriophage particles and agarose participated in the phase separation. These data suggests that phase separation is the cause of dilute agarose-induced increase in 0305φ8-36's propagation. Perhaps, phase separation also occurs in the wild and 0305φ8-36 is adapted to increase its anti-bacterial activity when in a weakly fluorescent-like phase. If phase separation can, in general, be used to increase anti-bacterial activity of virulent bacteriophages, phase separation has potential use during bacteriophage therapy of bacterial disease.

Phase separation is not caused when some other bacteriophages are substituted for 0305φ8-36. This observation supports the hypothesis that 0305φ8-36 is adapted to phase separation. In search of a means of adaptation, a significant segment of the 0305φ8-36 genome was found to encode proteins that are (1) clustered in the genome, (2) present in the virion and (3) sometimes characterized by the presence of domains typical of fibers: fibronectin type III fold and von Willebrand factor domain. These “extra” proteins are possibly present to conduct interactions with the environment, including phase partitioning.

Bacteriophage 0305φ8-36 also forms aggregates in situ (within plaques). The aggregates sometimes contain weakly fluorescent phase. Formation of aggregates may be a result of evolutionary selection for high host finding rate while opposing selection for high progeny production rate also occurs. Apparent consequences of a “trade-off” are observed via the following single-plaque observations: In clear plaques 4-5 cm in diameter, macroscopically visible, 1-2 mm opaque 0305φ8-36 aggregates form and sometimes concentrate at the plaque edge. The key observation is that a semi-clear ring beyond the plaque edge also sometimes forms, but only where an (opaque) aggregate is present. The following hypothesis is proposed: (1) The observed aggregation evolved to reduce progeny production at 0305φ8-36 concentrations high enough to threaten hosts with extinction. (2) Aggregate-associated bacteriophage particles eventually become more infective with time and seed bacteriophage growth in the semi-clear ring. In at least this case, “kill all winners”-based theories of bacteriophage-induced host evolution need revision. Bulls-eye (or target) plaque morphology is explained.

### Example 3

### In Situ Fluorescence Microscopy of Bacteriophage 0305φ8-36

Bacteriophage 0305φ8-36 is a large myovirus with unusual curly fibers (FIG. 5). The genome is 218,948 base pairs long; the tail is 486 nm long. The classical morphogenesis genes of the 8-36 genome are highly diverged and 8-36 has genes in a new category. The genes are called extra or other fiber. The designation “other fiber” comes from homology of some of these proteins with fibronectin 3 domains and von Willebrand factor domain.

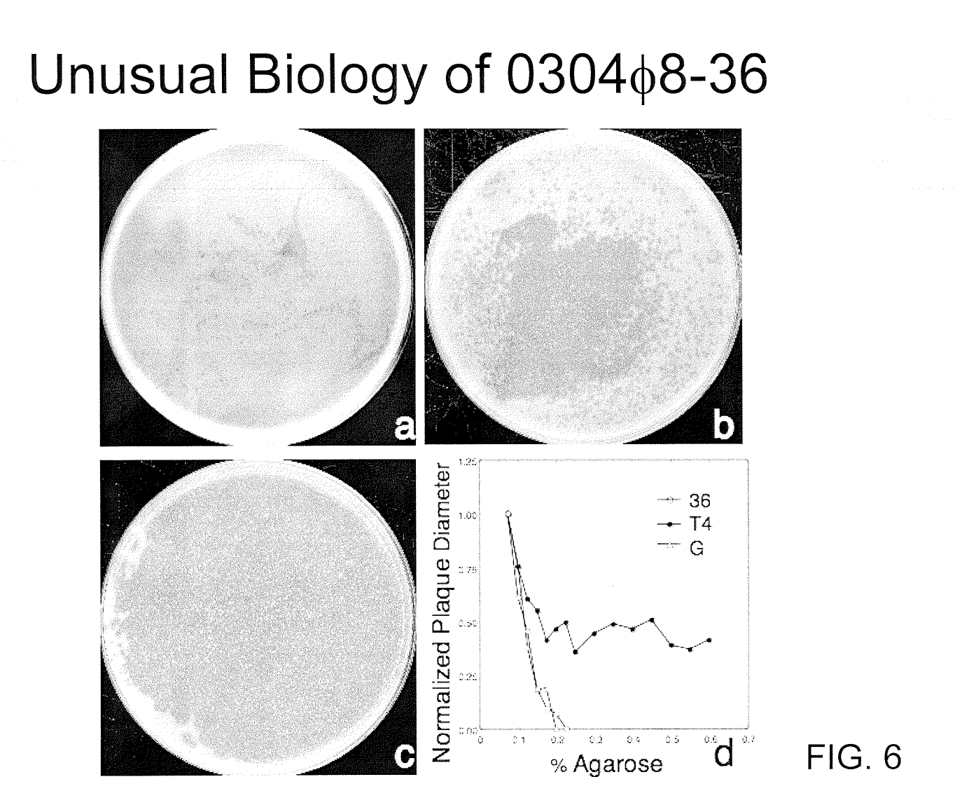

In addition to an unusual genome, 0305φ8-36 has unusual biology, including plaque radius vs. gel concentration plot that is a steep as the plot for bacteriophage G, the largest bacteriophage known (FIG. 6). 8-36 also does not clear liquid culture, though it does grow in liquid culture. In fact, 8-36 co-grows with the host and presumably co-evolves with the host, very unusual (though not unheard of) behavior. Furthermore, 8-36 undergoes aggregation and also a form of phase separation that has not yet been documented.

In spite of not clearing liquid culture, 8-36 form clear plaques and forms them comparatively quickly, within 8 hours. To determine what is in the plaque, a portion of a plaque was dissected with a pipet, stained with DAPI and then placed between a cover glass and microscope slide. Surprisingly, two phases one weakly fluorescent and the other comparatively dark. The existence of the two phases is confirmed by causing turbulent motion via pressing on the cover glass. The portion of the weakly fluorescent phase at the upper right is more flexible than the portion at the lower left; some bacteria are present. The smaller particles in the weakly fluorescent phase are moving and are either monomeric bacteriophage particles or, possibly, small aggregates. The presence of two phases requires the presence of bacteriophage 8-36 particles, bacteria and agarose. Surprisingly, the bacteriophage particles never cross the boundary between the two phases, even when motion is generated by pressing on the cover glass. Could this two-phase formation be the source of the increased host-clearing capacity?

If one focuses at the microscope slide, then one sees in addition, larger aggregates which sometimes have weakly fluorescent phase associated. The presence of the weakly fluorescent phase is confirmed by generating turbulent motion by pressing on the microscope slide. The aggregates get larger as the plaque ages.

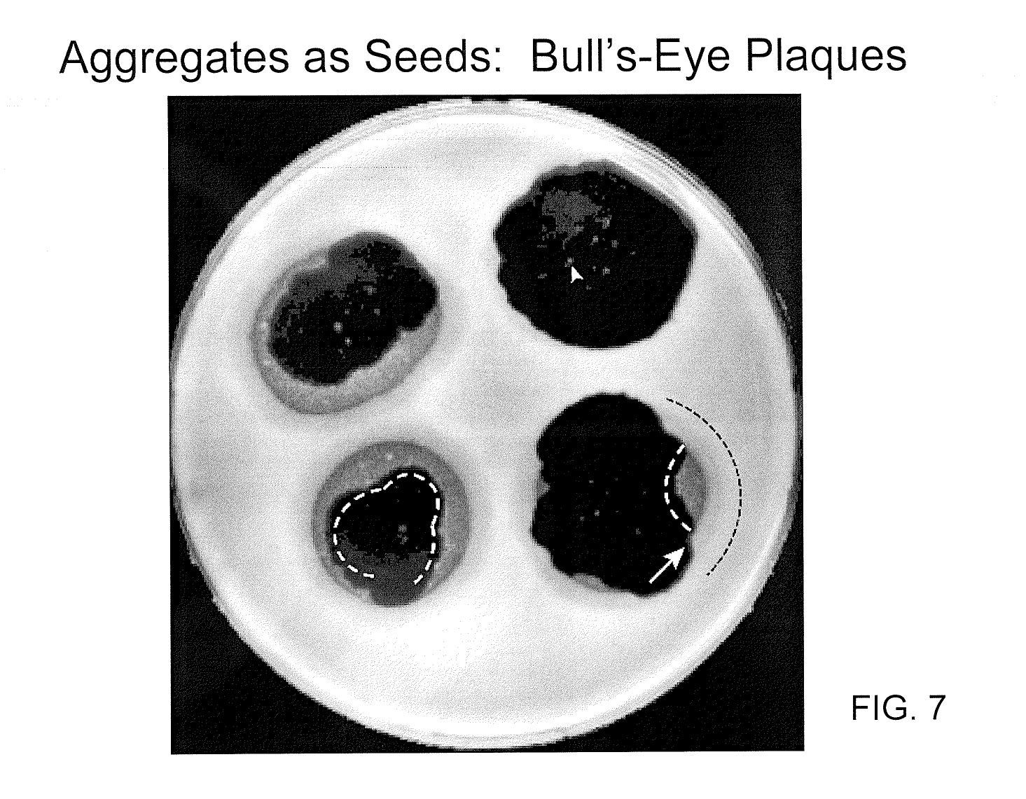

The older aggregates acquire elastic character and are also found exclusively at the surface of the microscopy slide where they sedimented at 1 g. Note that some aggregated particles are separated by a space that is possibly occupied by the tail and curly fibers, but some of these spaces are so long that something else must be present. These aggregates are so large that some become observable by eye (FIG. 7). In addition, pieces apparently break away from these large aggregates.

The smaller aggregates are observed and shown to be aggregates during drifting caused by capillary-induced fluid flow. The aggregation is so extensive that aggregates are observed macroscopically.

These aggregates are seen as either single opaque spots (arrowhead) or opaque zones of numerous spots (white dashed lines). Is aggregation a selected phenomenon? Aggregation might sequester the bacteriophage particles, thereby reducing their reproduction and increasing the future host finding rate. If so, then the aggregates should dissociate and seed additional bacteriophage growth. By 32 hours, some plaques also have rings. The bacteriophage in these rings is the original bacteriophage by both re-propagation and PFGE. Note that the position of the rings is correlated with the position of the aggregates. That is to say, the aggregates seed growth that forms the rings. How can the aggregates act at a distance when plenty of phage particles are between an aggregate and a ring. The aggregates must change state as they age. Empirically, the inter-particles distance decreases from 1.6±0.97 mm at 10.5 hr. to 0.54±0.4 mm at 32 hr. and 0.2±0.2 mm at 50 hr.

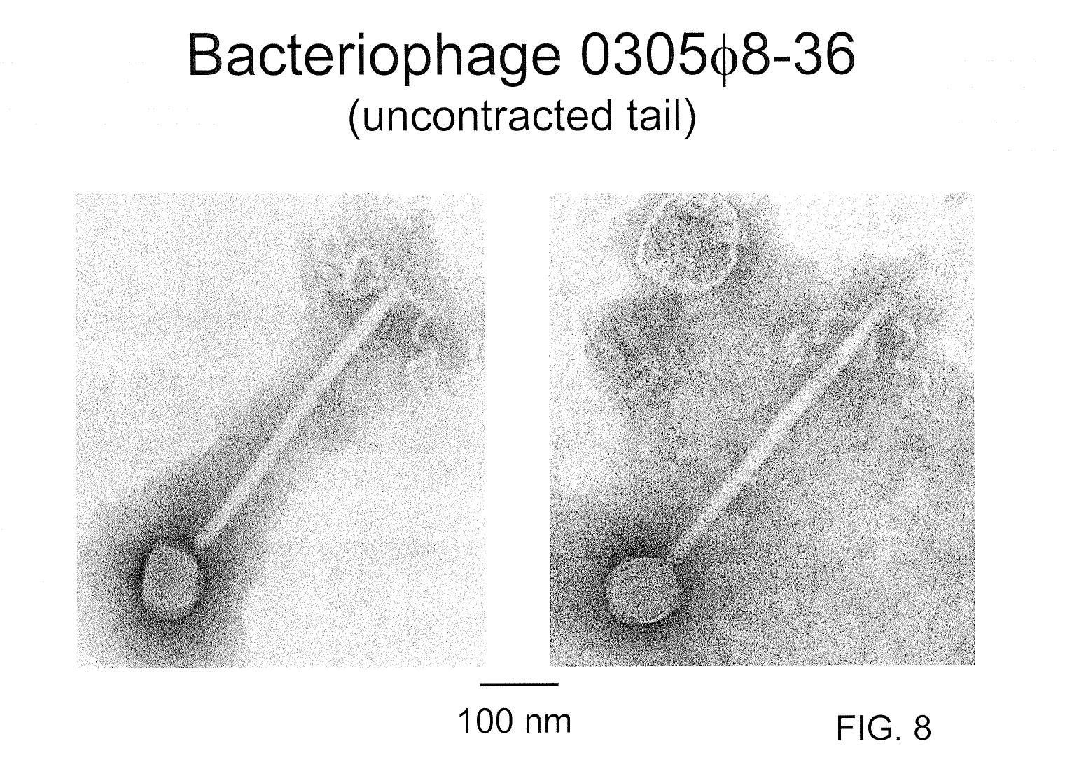

FIG. 8 shows an images of 8-36 with an uncontracted tail. Hints of extra fibers are seen.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,335,035
U.S. Pat. No. 6,586,493
U.S. Pat. No. 6,716,251
U.S. Patent Publn. 20050192249
U.S. Patent Publn. 20050267296
U.S. Patent Publn. 20060118418
U.S. Patent Publn. 20060280797
Ackermann and DuBow, In: Viruses of Prokaryotes, Volume I, CRC Press, Boca Raton, Fl, Chap. 3, 1987.
Ackermann et al., Can. J. Microbiol., 40:63-67, 1994.
American Type Culture Collection Catalogue Of Bacteria And Bacteriophages, 18^{th} Ed., 402-411, 1992.
Bustamante, Ann. Rev. Biophys. Biophys. Chem., 20:415-446, 1991.
Carlson, In: Bacteriophages: Biology and Applications, Kutter and Sulakvelidze (Eds.), 437-494, CRC Press, Fl, 2005.
Fangman, Nucleic Acids Res., 5:653-665, 1978.
Filippini et al., Appl. Environ. Microbiol., 72:4893-4898, 2006.
Goodman and Gilman's The Pharmacological Basis Of Therapeutics, Hardman et al. (Eds.), 7^{th} Ed.
Griess et al., Biopolymers, 28:1475-1484, 1989.
Grunewald and Cyrklaff, Curr. Opin. Microbiol., 9:437-442, 2006.
Jiang and Ludtke, Curr. Opin. Struct. Biol., 15:571-577, 2005.
Noble and Fuhrman, Aquat. Microb. Ecol., 14:113-118, 1998.
Physician's Desk Reference, 50^{th} ed., Medical Economics, 1996.
Remington's Pharmaceutical Sciences, 15^{th} Ed., Easton: Mack Publishing Co. 1405-1412 and 1461-1487, 1975.
Serwe et al., Biophys. J., 69:2649-2660, 1995.
Serwer and Wang, J. Nanosci. Nanotechnol., 5:2014-2028, 2005.
Serwer et al., In: Bacteriophages: Methods and Protocols, Clokie and Kropinski (Eds.), The Humana Press, Inc., Totowa, N.Y., 2007b.
Serwer et al., In: Rapid determination of genomic DNA length for new bacteriopages, Electrophoresis, 2007c (IN PRESS).
Serwer et al., J Microsc. 228:309-321, 2007d.
Serwer et al., J. Virol., 45(2):665-667, 1983.
Serwer et al., Virol. J, 4:21, 2007a.
Serwer et al., Virology, 329:412-424, 2004.
Serwer, J. Mol. Biol., 107(3):271-291, 1976.
The National Formulary XIV., 14^{th} Ed. Washington, American Pharmaceutical Association, 1975.
Wang et al., J. Microscop., 213:101-109, 2004.
Weinbauer, FEMS Microbiol. Rev., 28:127-181, 2004.
Williamson et al., Appl. Environ. Microbiol., 71:3119-3125, 2005.
Winter et al., Appl. Environ. Microbiol., 70:804-813, 2004.

What is claimed is:

1. A pharmaceutical composition comprising:
a) a bacteriophage; and
b) a polymer.

2. The pharmaceutical composition of claim 1, wherein the composition further comprises a bacteria or bacterial extract.

3. The pharmaceutical composition of claim 2, wherein the composition includes a bacteria that is a Staphylococcus, a Streptococcus, or Bacillus thuringiensis.

4. The pharmaceutical composition of claim 2, wherein the composition includes a bacterial extract derived from a Staphylococcus, a Streptococcus, or Bacillus thuringiensis.

5. The pharmaceutical composition of claim 1, wherein the polymer is agarose, a dextran, a cyclodextran, a copolymer of poly-N-isopropylacrylamide, a methylcellulose, a chitosan, a collagen, a tri-block copolymer of poly(ethylene glycol)-poly(lactic-co-glycolic acid)-poly(ethylene glycol), a tri-block copolymer of poly(propylene glycol)-poly(ethylene glycol)-poly (propylene glycol), poly(N-isopropyl acrylamide, hyaluronic acid, alginate, carboxymethylcellulose, polyvinyl pyrrolidone, polyvinyl alcohol, a polyethylene glycol, a water-soluble polyacylamide, a substituted polyacrylamide, a polydimethylacrylamide, a polyvinyl pyrrolidone, gelatin, polyvinyl alcohol, polylysine, or carageenan.

6. The pharmaceutical composition of claim 1, wherein the bacteriophage is 0305φ8-36.

7. A pharmaceutical composition comprising bacteriophage 0305φ8-36 and a pharmaceutically acceptable carrier.

8. A method of increasing the virulence of a bacteriophage, comprising contacting a bacteriophage with a composition comprising a polymer, wherein the virulence of the bacteriophage is increased.

9. The method of claim 8, wherein the composition further comprises a bacteria or bacterial extract.

10. The method of claim 8, wherein the composition undergoes a phase separation detectable by fluorescence microscopy after the bacteriophage is contacted with the composition.

11. The method of claim 9, wherein the composition comprises a bacterial extract.

12. The method of claim 9, wherein the composition comprises a bacterium.

13. The method of claim 11, wherein the bacterial extract is an extract of Staphylococcus epidermidis, Helicobacter pylori, Streptococcus mutans, Streptococcus oralis, Streptococcus parasanguis, Bacillus thuringiensis or Propionibacterium acnes.

14. The method of claim 12, wherein the bacterium is Staphylococcus epidermidis, Helicobacter pylori, Streptococcus mutans, Streptococcus oralis, Streptococcus parasanguis, Bacillus thuringiensis or Propionibacterium acnes.

15. The method of claim 8, wherein the polymer is selected from the group consisting of a polymer derived from agar, a dextran, a cyclodextran, a copolymer of poly-N-isopropylacrylamide, a methylcellulose, a chitosan, a collagen, a tri-block copolymer of poly(ethylene glycol)-poly(lactic-co-glycolic acid)-poly(ethylene glycol), a tri-block copolymer of poly(propylene glycol)-poly(ethylene glycol)-poly (propylene glycol), poly(N-isopropyl acrylamide, hyaluronic acid, alginate, carboxymethylcellulose, polyvinyl pyrrolidone, polyvinyl alcohol, a polyethylene glycol, a water-soluble polyacylamide, a substituted polyacrylamide, a polydimethylacrylamide, a polyvinyl pyrrolidone, gelatin, polyvinyl alcohol, polylysine, carageenan, and an analog thereof.

16. The method of claim 15, wherein the polymer is a polymer derived from agar.

17. The method of claim 16, wherein the polymer derived from agar is agarose.

18. The method of claim 8, wherein the concentration of polymer in the composition is about 0.001% to about 0.1%.

19. The method of claim 18, wherein the concentration of polymer in the composition is about 0.01% to about 0.05%.

20. The method of claim 19, wherein the concentration of polymer in the composition is about 0.075% to about 0.02%.

21. The method of claim 19, wherein the polymer is derived from agarose.

22. The method of claim 8, further comprising assessing virulence of the bacteriophage following contacting of the composition with the bacteriophage.

23. The method of claim 22, wherein virulence of the bacteriophage is assessed by measuring bacterial cell lysis.

24. The method of claim 22, wherein virulence is measured by assessing response of an animal or a plant with an infection to treatment following administration of the bacteriophage to the subject.

25. The method of claim 24, wherein the response of an animal is measured and the animal is a mammal.

26. The method of claim 25, wherein the mammal is a cow, a horse, a pig, a goat, or a human.

27. The method of claim 26, wherein the mammal is a cow with an infection further defined as bovine mastitis.

28. The method of claim 8, wherein virulence is increased relative to virulence of the bacteriphage prior to said contacting.

29. A method of isolating a therapeutic bacteriophage from a bacteriophage-containing sample, comprising the steps of:
a) placing a bacteriophage-containing sample on a surface;
b) contacting the bacteriophage-containing sample with a first composition comprising a polymer to form a lower layer;
c) contacting the lower layer with a second composition comprising a polymer and a host bacteria to form an upper layer on top of the lower layer;
d) removing a bacteriophage from a plaque that forms in the upper layer, wherein the plaque contains therapeutic bacteriophages.

30. The method of claim 29, wherein the method further comprises inoculating the host bacteria with bacteriophage by creating a passageway between the lower layer and the upper layer after step (c).

31. The method of claim 29, wherein the concentration of polymer in the first composition is about 0.1% to about 5.0%.

32. The method of claim 31, wherein the concentration of polymer in the first composition is about 0.001% to about 0.1%.

33. The method of claim 32, wherein the concentration of polymer in the first composition is about 0.01% to about 0.05%.

34. The method of claim 29, wherein the concentration of polymer in the second composition is about 0.001% to about 0.1%.

35. The method of claim 34, wherein the concentration of polymer in the second composition is about 0.01% to about 0.05%.

36. The method of claim 29, wherein the host bacteria is Staphylococcus epidermidis, Helicobacter pylori, Streptococcus mutans, Streptococcus oralis, Streptococcus parasanguis, Bacillus thuringiensis or Propionibacterium acnes.

37. The method of claim 29, wherein inoculating the host cells comprises piercing the second layer with a needle.

38. The method of claim 29, further comprising storing the plaque by freezing.

39. The method of claim 29, further comprising preparing a pharmaceutical composition comprising the bacteriphages of step e).

40. The method of claim 39, wherein the pharmaceutical composition can be applied in the treatment of a disease of a mammal or a plant with an infectious disease.

41. The method of claim 40, wherein the mammal is a cow, a horse, a pig, or a human.

42. The method of claim 41, wherein the mammal is a human.

43. The method of claim 42, wherein the mammal is a cow.

44. The method of claim 43, wherein the cow is a cow with bovine mastitis.

45. A method of treating or preventing an infectious disease in a subject, comprising contacting the subject with a pharmaceutically effective amount of a composition comprising a therapeutic bacteriophage that has been isolated in accordance with the method of claim 29.

46. The method of claim 45, wherein the composition further comprises a bacteria or bacterial extract.

47. The method of claim 45, wherein the composition further comprises a polymer.

48. The method of claim 47, wherein the polymer is derived from agarose.

49. The method of claim 45, wherein the subject is a mammal.

50. The method of claim 49, wherein the mammal is a human, a horse, a cow, or a pig.

51. The method of claim 50, wherein the mammal is a cow with bovine mastitis.

52. The method of claim 45, wherein the therapeutic bacteriophage is 0305φ8-36.

53. A method of treating or preventing an infectious disease in a subject, comprising contacting the subject with a composition comprising:
a) a bacteriophage; and
b) a polymer,
wherein the virulence of the bacteriophage is increased.

54. The method of claim 53, wherein the composition further comprises a bacteria or bacterial extract.

55. The method of claim 53, wherein the bacteria is Staphylococcus epidermidis, Helicobacter pylori, Streptococcus mutans, Streptococcus oralis, Streptococcus parasanguis, Bacillus thuringiensis or Propionibacterium acnes.

56. The method of claim 53, wherein the bacterial extract is derived from Staphylococcus epidermidis, Helicobacter pylori, Streptococcus mutans, Streptococcus oralis, Streptococcus parasanguis, Bacillus thuringiensis or Propionibacterium acnes.

57. The method of claim 53, wherein the polymer is selected from the group consisting of a polymer derived from agar, a dextran, a cyclodextran, a copolymer of poly-N-isopropylacrylamide, a methylcellulose, a chitosan, a collagen, a tri-block copolymer of poly(ethylene glycol)-poly(lactic-co-glycolic acid)-poly(ethylene glycol), a tri-block copolymer of poly(propylene glycol)-poly(ethylene glycol)-poly (propylene glycol), poly(N-isopropyl acrylamide, hyaluronic acid, alginate, carboxymethylcellulose, polyvinyl pyrrolidone, polyvinyl alcohol, a polyethylene glycol, a water-soluble polyacylamide, a substituted polyacrylamide, a polydimethylacrylamide, a polyvinyl pyrrolidone, gelatin, polyvinyl alcohol, polylysine, carageenan, and an analog thereof.

58. The method of claim 57, wherein the polymer is agarose.

59. The method of claim 58, wherein the composition comprises about 0.075% to about 0.02% agarose.

60. The method of claim 53, wherein the subject is a mammal.

61. The method of claim 60, wherein the mammal is a human, a cow, or a horse.

62. The method of claim 61, wherein the mammal is a cow with bovine mastitis.

63. The method of claim 53, wherein the bacteriophage is 0305φ8-36.
